(19) United States
(12) Patent Application Publication
(10) Pub. No.: US 2023/0077294 A1
(43) Pub. Date: Mar. 9, 2023

(54) MONITORING, SIMULATION AND CONTROL OF BIOPROCESSES

(71) Applicant: Sartorius Stedim Data Analytics AB, Umea (SE)

(72) Inventors: Christopher Peter McCready, Oakville (CA); Brandon Corbett, Oakville (CA); Rickard Sjögren, Umea (SE); Frida Nordström, Umea (SE)

(73) Assignee: Sartorius Stedim Data Analytics AB, Umea (SE)

(21) Appl. No.: 17/801,213

(22) PCT Filed: Jan. 14, 2021
(86) PCT No.: PCT/EP2021/050743

§ 371 Date: Aug. 19, 2022

Related U.S. Application Data
(63) Continuation-in-part of application No. 16/796,340, filed on Feb. 20, 2020, now Pat. No. 11,542,564.

(51) Int. Cl. G16B 5/00; C12M 1/36; C12M 1/34; G16B 40/20

(52) U.S. Cl. CPC G16B 5/00; C12M 41/48; C12M 41/30; G16B 40/20

(57) ABSTRACT

Methods for monitoring, controlling and simulating a bioprocess comprising a cell culture in a bioreactor are provided. The methods comprise obtaining values of one or more process conditions for the bioprocess at one or more maturities, and determining the specific transport rates of one or more metabolites in the cell culture using the values obtained as input to a machine learning model trained to predict the specific transport rates of the one or more metabolites at a latest maturity of the one or more maturities or a later maturity based at least in part on the values of one or more process conditions for the bioprocess at the one or more preceding maturities. The methods further comprise predicting one or more features of the bioprocess based at least in part on the determined specific transport rates. Systems, computer readable media and methods for providing tools to implement such methods are also provided.

### FIELD OF THE PRESENT DISCLOSURE

The present disclosure relates to computer implemented methods, computer programs and systems for the monitoring, simulation and control of bioprocesses. Particular methods, programs and systems of the disclosure use machine learning to predict one or more variables representative of the metabolic condition of cells in a bioprocess.

### BACKGROUND

Bioprocesses use biological systems to produce a specific biomaterial, for example a biomolecule with therapeutic effects. This process typically involves placing cells and/or microbes into a bioreactor with media containing nutrients under controlled atmospheric conditions. The media is consumed by the cells and used for growth and other metabolic functions, including production of the specific biomaterial and by-products.

The bioreactor typically contains or is associated with instrumentation that continuously (e.g., once every second, minute, hour) measures process conditions, such as temperature, pH, and dissolved oxygen, as well as addition of nutrients and gasses, and flow and content of streams leaving the bioreactor. Typically, samples of the culture are taken periodically (e.g., once or twice per day, or more) to measure the content of the bulk fluid, including the concentration of one or more metabolites (e.g., glucose, glutamine, lactate, NH4, etc.), the concentration of the product biomaterial (also referred to as titer), cell metrics such as total cells and viable cell density (VCD), and quality metrics of the product biomaterial (sometimes also called “quality attributes” or “critical quality attributes” (CQA), such as e.g. the glycosylation profile or activity of the product).

Statistical process analysis methods can be used to assess satisfactory performance of a bioprocess. In particular, multivariate statistical models (including principal component analysis—PCA—and (orthogonal) partial least square regression—(O)PLS) have become a popular tool for identifying process conditions that are important to ensure that CQAs are within specification (collectively referred to as “critical process parameters”, CPPs) and to establish acceptable ranges of these process conditions as a bioprocess progresses to completion. Such tools have been implemented in the Umetrics® software suite (Sartorius Stedim Data Analytics), which is a leading data analytics software for modeling and optimizing biopharmaceutical development and manufacturing processes.

In a typical bioprocess analysis, a series of process variables (e.g. a few dozen process variables including temperature, concentration of key nutrients and metabolites, pH, volume, gas concentrations, viable cell density, etc.) are measured during completion of the bioprocess. These process variables together represent the “process condition”. Many of these variables are highly correlated and as such methods such as PCA and PLS can be used to identify summary variables that capture the correlation structure in the data. These (typically relatively few) variables can then be extracted and the range of values of theses variables that define a “normal” process condition can be estimated. Further, models such as (O)PLS can be used to predict product titer from these process variables.

All of these approaches model the impact of process parameters on a product produced by cells without any understanding of how the process parameters impact the functioning of the cells and how this ultimately results in changes in CQAs and product titer. This lack of direct measurements of metabolic activity during process development creates a scenario wherein trial and error is used to design process operations. Using this approach, experiments are conducted, in an ad hoc manner, to determine conditions that produce high quality product with a relatively high yield. Further, this lack of understanding of the metabolic condition of the cells (which is responsible for process conditions resulting in particular CQAs and titer) means that diagnosing/predicting faults (e.g. abnormal metabolic operation) or optimizing a process require subject matter experts (SMEs) to come up with biologically relevant hypotheses for which metabolic conditions may underlie an observed behaviour.

Additionally, all of these approaches rely on an a posteriori analysis of the bioprocess and have limited ability to predict how a bioprocess will evolve. This means that they are ill-suited for implementing pre-emptive actions to correct the course of a bioprocess (rather than diagnose it as faulty after the fact, potentially wasting time and resources), and for building models that simulate the course of a bioprocess e.g. for process optimization purposes.

Therefore, a need exists for a system and method for improved methods for monitoring, simulating and controlling bioprocesses.

### SUMMARY

According to a first aspect of the disclosure, there is provided a computer-implemented method for monitoring a bioprocess comprising a cell culture in a bioreactor, the method including the steps of: obtaining values of one or more process conditions including one or more process parameters, one or more metabolite concentrations and/or one or more biomass-related metrics for the bioprocess at one or more maturities; determining the specific transport rates of one or more metabolites in the cell culture using the values obtained as input to a machine learning model trained to predict the specific transport rates of the one or more metabolites at a latest maturity of the one or more maturities or a later maturity based at least in part on the values of one or more process conditions for the bioprocess at the one or more maturities; and predicting one or more features of the bioprocess based at least in part on the determined specific transport rates.

The method of the first aspect may have any one or any combination of the following optional features.

Predicting one or more features of the bioprocess may comprise: comparing the specific transport rates or values derived therefrom to one or more predetermined values; and determining on the basis of the comparison whether the process is operating normally.

The one or more predetermined values may be specified as a function of maturity and comparing the specific transport rates or values derived therefrom to one or more predetermined values may comprise comparing the specific transport rates or values derived therefrom to one or more predetermined values associated with a maturity corresponding to the later maturity value.

Within the context of the present disclosure, maturity may be expressed in units of time. For example, maturity may refer to an amount of time since the start of a bioprocess or any other reference time. Thus, reference to particular “maturities” and “functions of maturity” should be interpreted to encompass “time points”, “times” and “functions of time”.

Obtaining the values of one or more process conditions may comprise directly or indirectly measuring the values of one or more process conditions. Obtaining the values of one or more process conditions may comprise receiving the values, for example from a user, computing device or memory.

The specific transport rate of a metabolite may be a specific consumption rate or a specific production rate. The specific transport rate of a metabolite i may be the net amount of the metabolite transported between the cells and the culture medium, per cell and per unit of maturity.

As will be described further below, predicting one or more features of the bioprocess comprises may comprise classifying the internal metabolic condition of the process between a plurality of classes, wherein the internal metabolic condition of the process includes the specific transport rates or values derived therefrom. Classifying the internal metabolic condition of the process may comprise classifying the internal metabolic condition of the process into an optimal or a suboptimal category for biomaterial production.

As will be described further below, predicting one or more features of the bioprocess comprises may comprise predicting a state trajectory of the process using one or more models of evolution of the process including the specific transport rates or variables derived therefrom. The one or more models may include a kinetic growth model and material balance equations. The state of the process may include the value of any of the variables in such models, such as e.g. the value of one or more cell culture parameters and/or metabolite concentrations. The state trajectory of the process may include the value of state variables at a plurality of time points/maturities.

As will be described further below, predicting one or more features of the bioprocess comprises may comprise predicting a future state of the process following a change in one or more process parameters, using one or more models of evolution of the process including the specific transport rates or variables derived therefrom.

As will be described further below, predicting one or more features of the bioprocess comprises may comprise predicting a current or future critical quality attribute (CDQ) associated with the process using one or more predictive models trained to predict the value of one or more CQAs based at least in apart on the specific transport rates or variables derived therefrom.

The one or more process conditions may include one or more process parameters selected from dissolved oxygen, dissolved CO2, pH, temperature, osmolality, agitation speed, agitation power, headspace gas composition (such as e.g. CO₂ pressure), flow rates (such as feed rate, bleed rate, harvest rate), feed medium composition and volume of the culture. The one or more process conditions may include one or more biomass related metrics selected from viable cell density, total cell density, cell viability, dead cell density, and lysed cell density. The one or more metabolite concentrations may include the concentration of one or more metabolites in the culture medium compartment. Unless the context indicates otherwise, metabolite concentrations typically refer to metabolite concentrations in the culture medium. The one or more values of process conditions used to predict the specific transport rates of the one or more metabolites may include at least one metabolite concentration value. The one or more values of process conditions may further include at least one further value of a process condition, preferably at least two further values. The one or more values of metabolite concentrations may include the concentration of one or more metabolites for which specific transport rates are determined. The one or more metabolites may instead or in addition include the concentration of one or more metabolites that are precursors of one or more metabolites for which specific transport rates are determined. The one or more metabolites may instead or in addition include the concentration of one or more metabolites that are products of reactions that produce or consume one or more metabolites for which specific transport rates are determined.

Predicting one or more features of the bioprocess comprising predicting the value of one or more critical quality attributes (CQAs) of the bioprocess using a predictive model that has been trained to predict CQAs using a set of predictor variables comprising the one or more specific transport rates. The predictive model may be a machine learning model, such as eg. a multivariate model. The predictive model may have been trained using data from a plurality of training runs.

The method may further comprise using a pre-trained predictive model to determine the value of one or more latent variables, wherein the predictive model is a linear model that uses process variables including the specific transport rates as predictor variables. In such embodiments, the step of comparing values derived from the specific transport rates to predetermined values may comprise comparing the value of one or more of the latent variables to corresponding predetermined values.

The pre-trained predictive model may have been trained to determine the value of one or more latent variables as a function of maturity. The predictive model may be a linear model that uses specific transport rates and optionally process conditions as predictor variables and maturity as a response variable. The predictive model may be a multivariate model such as PLS or OPLS model. The multivariate model may be a PLS model as defined in equations (1) and (2), where in equations (1) and (2): X is the m×n matrix of process variables at maturities m, Y is the m×1 matrix of maturity values, and T is the m×I matrix of score values that describe the aspects of the process variables that are most correlated with maturity, including the one or more latent variables.

The predictive model may be a principal component regression (PCR). The predictive model may be a PCR model where PCA is applied on the matrix X of process variables at maturities m, and the matrix Y of maturity values is regressed on the principal components thus obtained to identify the principal components most correlated with maturity, the PCA scores representing latent variables describing the aspects of the process variables that are most correlated with maturity.

The predictive model may have been pre-trained using data from a plurality of similar bioprocesses considered to operate normally, wherein a similar bioprocess is one that uses the same cells for the same purpose. The predictive model may have been pre-trained using data from a plurality of similar bioprocesses in which at least some of the bioprocesses differ from each other by one or more process conditions as a function of maturity.

The machine learning model may be a regression model. The machine learning model may be selected from a linear regression model, a random forest regressor, an artificial neural network (ANN), and a combination thereof.

Advantageously, the machine learning model is an ANN or an ensemble of ANNs. The present inventors have found that ANNs were particularly suited to the task at hand. Without wishing to be bound by theory, the inventors believe that this is at least in part because ANNs are well suited to predict values using input data that has a complex correlation structure. Thus, ANNs performed particularly well in predicting a plurality of specific transport rates jointly.

A linear regression model may be a partial least square or orthogonal partial least square model. The machine learning model may comprise a plurality of machine learning models, wherein each machine learning model has been trained to predict the specific transport rates of an individually selected subset of the one or more metabolites.

The machine learning model may have been trained to jointly predict the specific transport rates of the one or more metabolites at a later maturity based at least in part on the values of one or more process conditions for the bioprocess at one or more preceding maturities. Predicting the specific transport rates of a plurality of the metabolites (i.e. all or a subset thereof) jointly may advantageously increase the accuracy of the prediction where the specific transport rates of metabolites that are jointly predicted are correlated with each other. Without wishing to be bound by theory, it is believed that many metabolites will be associated with specific transport rates that are correlated at least to some extent due to the metabolites taking part in correlated metabolic pathways within the cell.

Obtaining values of one or more process conditions at one or more maturities may comprise obtaining values of the one or more process conditions at a plurality of maturities; and the machine learning model has been trained to predict the specific transport rates of the one or more metabolites at a latest of the plurality of maturities or a later maturity based at least in part on the values of one or more process conditions for the bioprocess at the plurality of maturities. The inventors have found that predicting the specific transport rates using predictor variables at a plurality of preceding maturities advantageously increased the accuracy of the predictions, compared to the use of predictor variables at a single preceding maturity. Without wishing to be bound by theory, it is believed that the accuracy of machine learning predictions are likely to be increased by using predictor variables at a plurality of time points because such data can capture information about the dynamics of the bioprocess.

Advantageously, the machine learning model may have been trained to predict the specific transport rates of the one or more metabolites at a latest of two distinct maturities or a later maturity based at least in part on the values of one or more process conditions for the bioprocess at the two distinct maturities. The inventors have found that predicting the specific transport rates using predictor variables at two preceding maturities advantageously increased the accuracy of the predictions, compared to the use of predictor variables at a single preceding maturity, but that further improvements in accuracy by including additional maturities were not as extensive. In other words, the inventors have found that much of the benefit associated with the use of a plurality of maturities could be obtained by using just two maturities.

The values of one or more process conditions used as input to the machine learning model may be associated with a plurality of maturities that are separated from each other by a difference in maturity that is approximately equal to the difference in maturity between the values used to train the machine learning model.

Predicting one or more features of the bioprocess may comprise determining the value of one or more variables derived from the specific transport rates by: using the specific transport rates to determine the concentration of the corresponding one or more metabolites at the later maturity, optionally wherein determining the concentration of the corresponding one or more metabolites at the later maturity comprises solving respective material balance equations.

In embodiments, the one or more metabolites comprise a desired product. As such, also described herein is a method of predicting the concentration of a desired product in a bioprocess.

Determining the concentration of a metabolite i at maturity k, where k is the maturity associated with the predicted specific transport rates, may comprise integrating any of equations (4), (4a)-(4f) and (28) between a preceding maturity at which mᵢ is known and maturity k. The method may further comprise determining the value of one or more variables derived from the specific transport rates by: using the specific transport rates to determine the concentration of the corresponding one or more metabolites at the later maturity, and using one of more of said concentrations to determine the value of a biomass related metric at the later maturity, optionally wherein determining the value of a biomass related metric at the later maturity comprises solving a kinetic growth model. Solving a kinetic growth model at maturity k may comprise integrating any of equations (14) to (17) between a preceding maturity at which xᵥ, xₗ, x_{d} and/or xₜ are known and maturity k.

In embodiments, the biomass related metric comprises an amount of biomass. In some such embodiments, the biomass is a desired product of the bioprocess.

The method may further comprise using one or more of the said metabolite concentrations and/or biomass related metric values as inputs to the machine learning model to predict specific transport rates at a further maturity. In such embodiments, the method may be used to simulate the bioprocess, by iteratively determining the values of the process conditions used as inputs to the machine learning model to predict specific transport rates at subsequent maturities, and determining metabolite concentrations and biomass related metrics from the subsequent maturities.

As such, also described according to the present aspect are methods of simulating a bioprocess comprising a cell culture in a bioreactor, the methods comprising: obtaining initial values of one or more process conditions including one or more process parameters, one or more metabolite concentrations and/or one or more biomass-related metrics for the bioprocess at one or more initial maturities; determining the specific transport rates of one or more metabolites in the cell culture using the values obtained as input to a machine learning model trained to predict the specific transport rates of the one or more metabolites at a latest maturity of the one or more maturities or a later maturity based at least in part on the values of one or more process conditions for the bioprocess at the one or more maturities; predicting values of one or more process conditions including one or more process parameters, one or more metabolite concentrations and/or one or more biomass-related metrics for the bioprocess at based at least in part on the determined specific transport rates; and repeating the steps of determining and predicting using the predicted values of the one or more process conditions.

Values for any process parameters used as input to the machine learning model may be set to the corresponding initial values (i.e. assuming that said process parameters remain constant). Instead or in addition to this, values for any process parameters used as input to the machine learning model may be obtained from a user interface, computing device or memory. For example, trajectories for one or more process parameters may be provided as input to the method, wherein a trajectory for a process parameter comprises values for the process parameter at a plurality of maturities.

The methods of the present aspect may be performed using process condition values that are received from a user, computing device or memory. The methods of the present aspect may be performed using process condition values acquired in real time during the operation of a bioprocess. Such process condition values may comprise operational settings (i.e. parameters that are set by an operator), and/or values that are measured inline or offline during operation of the bioprocess. Therefore, the methods of monitoring a bioprocess described herein may be implemented in real time, i.e. during operation of the bioprocess. In such embodiments, the step of obtaining values of one or more process conditions may comprise receiving values for the one or more process conditions at the latest maturity for which such values have been measured or determined, and optionally obtaining values of the one or more process conditions at one or more preceding maturities from a data store.

Any of the methods of the present aspect may comprise recording the values of one or more process conditions, the determined specific transport rates and/or values derived therefrom in a data store.

The methods may further comprise outputting a signal to a user if the comparison step indicates that the bioprocess is not operating normally. A signal may be output through a user interface such as a screen, or through any other means such as audio or haptic signaling.

Comparing the value(s) of the one or more specific transport rates or variables derived therefrom to one or more predetermined values may comprise comparing the value(s) of the one or more specific transport rates or variables derived therefrom to the average value for the corresponding variables in a set of bioprocesses that are considered to operate normally. A bioprocess may be considered to operate normally if the value(s) of the one or more specific transport rates or variables derived therefrom is/are within a predetermined range of the average value for the respective corresponding variable in a set of bioprocesses that are considered to operate normally. The predetermined range may be defined as a function of the standard deviation associated with the average value of the respective corresponding variables across the set of bioprocesses considered to operate normally. A bioprocess may be considered to operate normally if the value of one or more variables t is within a range defined as average(t)±n*SD(t), where average(t) is the average value of the variable tin a set of bioprocesses that are considered to operate normally, SD(t) is the standard deviation associated with average(t), and n is a predetermined constant (which may be the same for the subrange average(t)+n*SD(t) and for the subrange average(t)−n*SD(t), or may differ between these subranges). In embodiments, n is 1, 2, 3 or a value that results in a chosen confidence interval, for example a 95% confidence interval. In embodiments, a bioprocess may be considered to operate normally if the value of one or more variables t is within a range defined as a confidence interval, e.g. a 95% confidence interval, around average(t), based on an assumed distribution of t. An assumed distribution may be a Gaussian (normal) distribution, a chi-squared distribution, etc. Where the assumed distribution is a normal distribution, a p % confidence interval (where p can be e.g. 95) may be equivalent to a range of average(t)±n*SD(t), where n is a single value that results in the p % confidence interval (e.g. n may be about 1.96 for a 95% confidence interval).

All of the steps of the methods described herein are computer implemented unless context indicates otherwise. In particular, any of the steps of the present method may be implemented by a computing device, optionally in operable communication with one or more sensors, other computing devices and/or user interfaces.

The methods may further comprise predicting the effect of a particular value of a process parameter at a later maturity by including the particular value in the material balance equations, the kinetic growth model and/or the input values used by the machine learning model to predict specific transport rates at a further maturity.

According to a second aspect, there is provided a computer implemented method for controlling a bioprocess comprising a cell culture in a bioreactor, the method comprising: implementing the steps of any of the embodiments of the first aspect; comparing the specific transport rates or values derived therefrom to one or more predetermined values; determining on the basis of the comparison whether to implement a corrective action; and sending a signal to one or more effector device(s) to implement a corrective action if the determining step indicates that a corrective action is to be implemented.

The method according to the present aspect may have any of the features disclosed in relation to the first aspect. The method of the present aspect may further have any one or any combination of the following optional features.

Determining on the basis of the comparison whether to implement a corrective action may comprise determining on the basis of the comparison whether the bioprocess is operating normally, and sending a signal to one or more effector device(s) to implement a corrective action if the comparison step indicates that the bioprocess is not operating normally.

Determining on the basis of the comparison whether to implement a corrective action may comprise determining on the basis of the comparison whether the bioprocess is operating optimally, and sending a signal to one or more effector device(s) to implement a corrective action if the comparison step indicates that the bioprocess is not operating optimally.

Determining whether the bioprocess is operating optimally may comprise determining whether a different set of one or more process conditions is associated with improved specific transport rates or values derived therefrom. In such embodiments, the one or more predetermined values may comprise values associated with one or more different sets of process conditions.

The method may further comprise repeating the steps of the method of monitoring the bioprocess, after a predetermined period of time has elapsed since obtaining the preceding measurements.

A corrective action may be associated with a change in the value of one or more process conditions. The method may further comprise determining a corrective action to be implemented by predicting the effect of the corrective action by including the value in the material balance equations, the kinetic growth model and/or the input use by the machine learning model to predict specific transport rates at a further maturity.

An effector device may be any device coupled to the bioreactor and which is configured to change one or more physical or chemical conditions in the bioreactor.

According to a third aspect, there is provided a method of optimizing a bioprocess comprising a cell culture in a bioreactor, the method comprising performing the method of any embodiment of the preceding aspect using a first set of process conditions and at least a further set of process conditions and determining whether the further set of process conditions is preferable to the first set of process conditions by comparing the specific transport rates or values derived therefrom associated with the respective sets of process conditions.

The method may further comprise selecting a further set of process conditions, and comparing the specific transport rates or values derived therefrom associated with the further set of process conditions with those associated with one or more previously used sets of conditions.

The step of selecting a further set of process conditions may comprise receiving a further set of conditions from a user interface, obtaining a further set of conditions from a database or computing device, using an optimization algorithm to determine a further set of conditions, or combinations thereof.

Also disclosed, according to a fourth aspect, is a method of providing a tool for monitoring a bioprocess comprising a cell culture in a bioreactor, the method including the steps of: obtaining measurements of one or more process conditions including one or more process parameters, one or more metabolite concentrations and/or one or more biomass-related metrics for a plurality of bioprocess at a plurality of maturities; determining the specific transport rates of one or more metabolites in the cell culture at the plurality of maturities using a material balance model and said measurements; using the measurements and corresponding determined specific transport rates to train a machine learning model trained to predict the specific transport rates of the one or more metabolites at a latest of one or more maturities or a later maturity based at least in part on the values of one or more process conditions for the bioprocess at the one or more maturities. The method may comprise any of the features described in relation to the first aspect.

According to a fifth aspect, there is provided a system for monitoring a bioprocess comprising a cell culture in a bioreactor, the system including: at least one processor; and at least one non-transitory computer readable medium containing instructions that, when executed by the at least one processor, cause the at least one processor to perform operations comprising: obtaining values of one or more process conditions including one or more process parameters, one or more metabolite concentrations and/or one or more biomass-related metrics for the bioprocess at one or more maturities; determining the specific transport rates of one or more metabolites in the cell culture using the values obtained as input to a machine learning model trained to predict the specific transport rates of the one or more metabolites at a latest maturity of the one or more maturities or a later maturity based at least in part on the values of one or more process conditions for the bioprocess at the one or more maturities; and predicting one or more features of the bioprocess based at least in part on the determined specific transport rates.

The system according to the present aspect may be configured to implement the method of any embodiment of the first aspect. In particular, the at least one non-transitory computer readable medium may contain instructions that, when executed by the at least one processor, cause the at least one processor to perform operations comprising any of the operations described in relation to the first aspect.

The system may further comprise, in operable connection with the processor, one or more of: a user interface, wherein the instructions further cause the processor to provide, to the user interface for outputting to a user, one or more of: the value of the one or more specific transport rates or variables derived therefrom, the result of the comparison step, and a signal indicating that the bioprocess has been determined to operate normally or to not operate normally; one or more biomass sensor(s); one or more metabolite sensor(s); one or more process condition sensors; and one or more effector device(s).

According to a sixth aspect of the disclosure, there is provided a system for controlling a bioprocess, the system including:
a system for monitoring a bioprocess according to the preceding aspect; and
at least one effector device operably connected to the processor of the system for monitoring a bioprocess.

The system according to the present aspect may be configured to implement the method of any embodiment of the second aspect. In particular, the at least one non-transitory computer readable medium may contain instructions that, when executed by the at least one processor, cause the at least one processor to perform operations comprising any of the operations described in relation to the second aspect.

According to a seventh aspect, there is provided a system for providing a tool for monitoring a bioprocess comprising a cell culture in a bioreactor, the system including: at least one processor; and at least one non-transitory computer readable medium containing instructions that, when executed by the at least one processor, cause the at least one processor to perform operations comprising:

obtaining measurements of one or more process conditions including one or more process parameters, one or more metabolite concentrations and/or one or more biomass-related metrics for a plurality of bioprocess at a plurality of maturities; determining the specific transport rates of one or more metabolites in the cell culture at the plurality of maturities using a material balance model and said measurements; using the measurements and corresponding determined specific transport rates to train a machine learning model trained to predict the specific transport rates of the one or more metabolites at a latest of one or more maturities or a later maturity based at least in part on the values of one or more process conditions for the bioprocess at the one or more maturities.

The system according to the present aspect may be configured to implement the method of any embodiment of the fourth aspect. In particular, the at least one non-transitory computer readable medium may contain instructions that, when executed by the at least one processor, cause the at least one processor to perform operations comprising any of the operations described in relation to the fourth aspect. According to a further aspect, there is provided a computer-implemented method using a cell metabolism state observer for predicting an amount of at least one biomaterial produced or consumed by a biological system in a bioreactor is provided. The method includes:
measuring process conditions including one or more process parameters, one or more metabolite concentrations and one or more biomass-related metrics for the biological system as a function of time;
determining specific transport rates for the biological system, including specific consumption rates of metabolites and specific production rates of metabolites, using the measured process conditions as input to a machine learning model trained to predict the specific transport rates of the one or more metabolites at a latest of one or more times or a later time based at least in part on the values of one or more process conditions for the bioprocess at the one or more times;
providing the process conditions and the specific transport rates to a hybrid system model configured to predict production of the biomaterial, the hybrid system model comprising:
a kinetic growth model configured to estimate cell growth as a function of time; and
a metabolic condition model based on metabolite specific consumption or secretion rates, select process conditions, wherein the metabolic condition model is configured to classify the biological system into an internal metabolic condition; and
predicting an amount of the biomaterial based on the hybrid system model.

The kinetic growth model may be configured to estimate viable cell density. The kinetic growth model may be configured to account for lysed cells. In embodiments, the hybrid model is used as a state observer, and provides an estimate of an internal metabolic state of the biological system and an estimate of the cell state of the biological system. In embodiments, the kinetic growth model is used as a state observer and provides an estimate of the cell state of the biological system. The method may comprise: obtaining a current measurement of the metabolite; determining a consumption rate for the metabolite using the machine learning model; and predicting a future concentration of the metabolite using the metabolic state observer and the current measurement.

The method may further comprise: classifying the internal metabolic condition into an optimal or a suboptimal category for biomaterial production; and sending a notification to a user, when the internal metabolic condition is classified into a suboptimal category.

The kinetic growth model may comprise a Monod kinetic model or a saturation kinetic model. In embodiments, cell density or cell viability for the biological system may be measured as a function of time. The kinetic growth model may further be configured to estimate microbial cell growth as a function of time.

The metabolic condition model may comprise one or more of a machine learning model, a deep learning model, a principal component analysis (PCA) model, a partial least squares (PLS) model, a partial least squares discriminant analysis (PLS-DA) model, or an orthogonal partial least squares discriminant analysis (OPLS-DA) model.

In embodiments, a test sample may be obtained from the bioreactor, and the method may determine whether the amount of the biomaterial in the test sample is within a range predicted by the hybrid system model.

Parameters of the hybrid system model may be updated when the hybrid system model is in operation. Parameters of the hybrid system may include coefficients associated with the hybrid system model.

According to any aspect, process conditions may include one or more of pH, temperature, dissolved oxygen, osmolality, process flow leaving the bioreactor, growth media, by-products, amino acids, metabolites, oxygen flow rate, nitrogen flow rate, carbon dioxide flow rate, air flow rate, and agitation rate. The growth media or feed may comprise nutrients including an amino acid, a saccharide, or an organic acid. The by-products of the bioprocess may include an amino acid, a saccharide, an organic acid, or ammonia.

The method of the present aspect may further comprise: determining optimal process conditions for the bioreactor based on the hybrid system model; measuring experimental process conditions of the bioreactor using one or more sensors as a function of time; monitoring the measured experimental process conditions to detect deviations from the optimal process conditions; and when a deviation is detected, sending a notification to a user.

The method may further comprise: determining optimal process conditions for the bioreactor based on the hybrid system model; measuring experimental process conditions of the bioreactor using one or more sensors as a function of time; monitoring the measured experimental process conditions to detect deviations from the optimal process conditions; and providing feedback to a controller controlling the bioreactor to automatically adjust the experimental process conditions to minimize deviation from the optimal process conditions.

The method may further comprise: determining optimal process conditions for the bioreactor based on the hybrid system model; measuring experimental process conditions of the bioreactor using one or more sensors as a function of time; monitoring the measured experimental process conditions to detect deviations from the optimal process conditions; sending a notification to a user when a deviation is detected; and providing feedback to a controller controlling the bioreactor to automatically adjust the experimental process conditions to minimize deviation from the optimal process conditions.

The method may further comprise: simulating, using the hybrid system model, a predicted amount of at least one biomaterial, wherein the hybrid system model is initialized with the process conditions; and determining one or more states of the biological system based on the simulation.

The method may further comprise adjusting the process conditions based on an optimization method to determine a set of process conditions that optimize predicted trajectories, product quantity (titer), and/or product quality.

The method may comprise calibrating a hybrid system model for predicting a biomaterial produced in a bioreactor by a biological system comprising: obtaining experimental data including measurement of one or more process conditions comprising one or more process parameters, one or more metabolite concentrations, and one or more biomass-related metrics (such as e.g. a cell amount) for a plurality of bioreactor batches, each batch associated with a specific set of process conditions; determining a growth rate under ideal conditions, using a kinetic model of the hybrid system model, based on the experimental data; determining a cell lysis parameter, using the kinetic model, based on the growth rate under the ideal conditions and the growth rate from the experimental data; determining specific production rates or specific consumption rates of metabolites using material balance equations and the experimental data; determining kinetic parameters for factors that inhibit growth to minimize differences between the growth rate under the ideal conditions and the growth rate from the experimental data; training a machine learning model predict specific production/consumption rates; and training a metabolic condition model of the hybrid system model for classification of the biological system into a metabolic state associated with specific productivity of the biomaterial produced by the biological system, based on measured specific consumption or measured secretion rates of the metabolites.

A set of parameters may be provided to an optimization module, wherein the set of parameters includes the growth rate under the ideal conditions, the specific consumption rates and the specific production rates of metabolites, and new process conditions, to determine process conditions to optimize production of the biomaterial.

The method may comprise monitoring bulk properties of the bioreactor using principal component analysis (PCA). An output of the bioreactor may be predicted using partial least squares (PLS) regression. The output may be an amount of the biomaterial. A “biomaterial” may include a metabolite, a cell, a desired protein, an antibody, an immunoglobulin, a toxin, one or more by-products, a target molecule, or any other type of molecule manufactured using a bioreactor. There may be more than one biomaterial of interest including the product, a target biologic.

As used herein, “factors that inhibit growth” may include a substrate limitation, a temperature or pH shift, or metabolites that inhibit growth. The term “specific productivity” may refer to the amount of product produced on a per cell basis. The term “process optimization” may refer to determining optimal adjustments or settings for a process. Metabolites may include any suitable analyte, including but not limited to: amino acids (e.g., alanine, arginine, aspartic acid, asparagine, cysteine, cysteine, glutamic acid, glutamine, glycine, histidine, hydroxyproline, isoleucine, leucine, lysine, methionine, phenylalanine, proline, serine, threonine, tryptophan, tyrosine, valine, etc.), saccharides (e.g., fucose, galactose, glucose, glucose-1-phosphate, lactose, mannose, raffinose, sucrose, xylose, etc.), organic acids (e.g., acetic acid, butyric and 2-hydroxy-butyric acids, 3-hydroxybutyric acid, citric acid, formic acid, fumaric acid, isovaleric acid, lactic acid, maleic acid, propionic acid, pyruvic acid, succinic acid, etc.), other organic compounds (e.g., acetone, ethanol, pyroglutamic acid, etc.).

According to a further aspect, there is provided a non-transitory computer readable medium comprising instructions that, when executed by at least one processor, cause the at least one processor to perform the method of any embodiment of any aspect described herein.

According to a further aspect, there is provided a computer program comprising code which, when the code is executed on a computer, causes the computer to perform the method of any embodiment of any aspect described herein.

### BRIEF DESCRIPTION OF THE DRAWINGS

Embodiments of the present disclosure will now be described by way of example with reference to the accompanying drawings in which:

FIG. 1 shows a simplified process diagram for a generic bioprocess according to embodiments of the present disclosure;

FIG. 2 is a flowchart illustrating a method of providing a tool according to embodiments of the present disclosure; in particular, the flowchart illustrates a model calibration procedure, which results in a calibrated model that can be used to predict one or more metabolic condition variables of a bioprocess;

FIG. 3 is a flowchart illustrating a method of predicting one or more metabolic condition variables according to embodiments of the present disclosure; in particular, the flowchart illustrates a model deployment procedure through which metabolic condition variables of a bioprocess can be predicted;

FIG. 4 illustrates the impact of selected parameters on variables of a kinetic growth model according to embodiments of the present disclosure; FIG. 4A is a plot showing the value of a correction factor (y-axis) capturing the effect of a growth inhibition variable on growth rate as a function of the value of the growth inhibiting variable (x-axis, e.g. concentration of a metabolite that has a cytostatic effect) for three exemplary values of the parameter θ_{i,n} which in this example represents the approximate value of the variable zₙ above which the variable starts to inhibit growth; FIG. 4B is a plot showing the value of a correction factor (y-axis) capturing the effect of a substrate limiting variable on growth rate as a function of the value of the substrate limiting variable (x-axis, e.g. concentration of a metabolite such as a nutrient) for three exemplary values of the parameter θ_{s,n} which in this example represents the approximate value of the variable zₙ below which the variable starts to have a limiting effect on growth; FIGS. 4C and 4D are a plots showing the value of a correction factor (y-axis) capturing the effect of a variable that has a quadratic effect on growth as a function of the value of the of the quadratic effect variable (x-axis, e.g. temperature, pH) for three exemplary values of the parameter parameter θ_{q,n} at a fixed value of μ_{q,n} (D) and three exemplary values of the parameter μ_{q,n} at a fixed value of θ_{q,n} (C), where in these examples parameter θ_{q,n} represents the spread of the effect and parameter μ_{q,n} represents the value at which maximum growth occurs;

FIG. 5 illustrates schematically a system according to embodiments of the present disclosure;

FIG. 6 illustrates schematically a computing architecture for implementing a method according to embodiments of the disclosure;

FIG. 7 shows a flowchart of an exemplary method of monitoring a bioprocess using a hybrid model as described herein;

FIG. 8 shows a flowchart of an exemplary method of simulating a bioprocess using a hybrid model as described herein;

FIGS. 9A-D show examples of the use of a hybrid model as described herein; FIGS. 9A-C show examples of the impact of independent variable adjustments to the growth profile according to the present disclosure: for each figure, the predicted profiles for growth states are shown in solid lines and associated data for measured states is included for comparison; (A) example of the measured and predicted trajectories for a batch with a temperature shift (inhibits growth), (B) measured and predicted trajectories for a batch with a pH shift and a low feed rate (glucose depletion), (C) measured and predicted trajectories for a batch with a pH and temperature shift; FIG. 9D show example outputs of a cell state classification by the hybrid model, according to the present disclosure; the figure shows a PCA score scatter plot providing observability of a metabolic disturbance caused by a depletion in glucose (e.g., predictions from the state observer using PCA); Glucose concentrations within the circle centered at the origin represent normal operation—glucose values outside of this range indicate increased risk of reduction in titer or product quality issues;

FIG. 10 shows the results of a bioprocess monitoring/control process according to an exemplary embodiment of the present disclosure (A) and comparisons between predictions obtained using various implementations according to the disclosure (B-D); (A) each plot compares, for a respective metabolite: (i) on the left, the mean square error (MSE) between (a) the specific consumption/secretion rate predicted by a metabolic neural network for the metabolite and (b) the corresponding specific consumption/secretion rate calculated retrospectively from measurements of the respective metabolite concentration (where the height of the bar represents the average MSE over the 6 folds of a model cross-validation process, and the error bars indicate the standard deviation across the 6 folds); and (ii) on the right, the MSE between (a) the specific consumption/secretion rate calculated for the respective metabolite as the average across values calculated from retrospective metabolic concentration measurements across 12 batches and (b) the corresponding specific consumption/secretion rate calculated retrospectively from measurements of the respective metabolite concentration (where the height of the bar represents the average MSE over the 12 batches and the error bars indicate the standard deviation around this average); note that the “baseline” data is calculated using metrics derived from the same measurements that were used as ground truth for the calculation of the MSE and is therefore artificially low; (B-D) each plot compares, for a respective metabolite, the mean square error (MSE) between (a) the specific consumption/secretion rate predicted by a metabolic neural network for the metabolite and (b) the corresponding specific consumption/secretion rate calculated retrospectively from measurements of the respective metabolite concentration (where the height of the bar represents the average MSE over the 6 folds of a model cross-validation process, and the error bars indicate the standard deviation across the 6 folds); (B) the left bar shows the data for a metabolic neural network using a lag=0, the middle bar shows the data for a metabolic network using a lag=1 (as is the case for the network used to obtain the predictions on FIG. 10A), and the right bar shows the data for a metabolic network using a lag=2; (C) the left bar shows the data for a metabolic neural network using only metabolite concentrations as input values, and the right bar shows the data for a metabolic neural network using all available variables as input values (as is the case for the network used to obtain the predictions on FIG. 10A); (D) the left bar shows the data for a metabolic neural network using only metabolite concentrations as input values (as is the case for the network used to obtain the left bar predictions on FIG. 10C), and the right bar shows the data for a metabolic neural network using as input values 4 out of the 5 metabolite concentrations used by the network that generated the data for the left bar (all except for glucose concentrations);

FIG. 11 shows the results of a bioprocess monitoring/control process according to an exemplary embodiment of the present disclosure: the plot compares (i) on the left, the MSE between the specific production rates (SPR) predicted by a titer neural network and the corresponding SPRs calculated from retrospectively measured product concentrations (where the height of the bar represents the average MSE over the 6 folds of a network cross-validation process, and the error bars indicate the standard deviation across the 6 folds); and (ii) on the right, the MSE between the SPRs calculated as averages across 12 batches and the SPRs calculated from retrospectively measured product concentrations (where the height of the bar represents the average MSE over the 12 batches and the error bars indicate the standard deviation around this average); again, the “baseline” data is calculated using metrics derived from the same measurements that were used as ground truth for the calculation of the MSE and is therefore artificially low;

FIG. 12 shows the results of a bioprocess simulation according to an exemplary embodiment of the present disclosure; each plot relates to a metabolite that is represented in the kinetic growth model used for the simulation, and shows: (i) on the left: the mean square error (MSE) between (a) the concentration of the respective metabolite predicted by a kinetic growth model using a metabolic neural network to predict specific transport rates for each of glucose, glutamine, lactate, glutamate and ammonia at each time point and (b) the corresponding (retrospectively) measured concentration (where the height of the bar represents the average MSE over 6 folds of a 6-fold model cross-validation process, and the error bars indicate the standard deviation across the 6 folds); and (ii) on the right, the MSE between (a) the concentrations of the respective metabolite calculated by the same kinetic growth model but using the average specific transport rates calculated across 12 batches from (retrospectively) measured concentrations and (b) the corresponding (retrospectively) measured concentrations (where the height of the bar represents the average MSE over the 12 batches and the error bars indicate the standard deviation around this average); note that the “baseline” data is calculated using metrics derived from the same measurements that were used as ground truth for the calculation of the MSE and is therefore artificially low.

Where the figures laid out herein illustrate embodiments of the present invention, these should not be construed as limiting to the scope of the invention. Where appropriate, like reference numerals will be used in different figures to relate to the same structural features of the illustrated embodiments.

### DETAILED DESCRIPTION

Specific embodiments of the invention will be described below with reference to the figures.

Bioprocess

As used herein, the term “bioprocess” (also referred to herein as “biomanufacturing process”) refers to a process where biological components such as cells, parts thereof such as organelles or multicellular structures such as organoids or spheroids are maintained in a liquid medium in an artificial environment such as a bioreactor. In embodiments, the bioprocess refers to a cell culture. A bioprocess typically results in a product, which can include biomass and/or one or more compounds that are produced as a result of the activity of the biological components. A bioreactor can be a single use vessel or a reusable vessel in which a liquid medium suitable for carrying out a bioprocess can be contained. Example bioreactor systems suitable for bioprocesses are described in US 2016/0152936 and WO 2014/020327. For example, a bioreactor may be chosen from: advanced microbioreactors (such as e.g. Ambr® 250 or Ambr® 15 bioreactors from The Automation Partnership Ltd.), single use bioreactors (e.g. bag-based bioreactors such as Biostat® STR bioreactors from Sartorius Stedim Biotech GmbH), stainless steel bioreactors (such as e.g. 5 to 2,000 I bioreactors available in the Biostat® range from Sartorius Stedim Systems GmbH), etc. The present invention is applicable to any type of bioreactor and in particular to any vendor and any scale of bioreactor from benchtop systems to manufacturing scale systems.

A cell culture refers to a bioprocess whereby live cells are maintained in an artificial environment such as a bioreactor. The methods, tools and systems described herein are applicable to bioprocesses that use any types of cells that can be maintained in culture, whether eukaryotic or prokaryotic. The invention can in particular be used to monitor and/or control bioprocesses using cells types including but not limited to mammalian cells (such as Chinese hamster ovary (CHO) cells, human embryonic kidney (HEK) cells, Vero cells, etc.), non-mammalian animal cells (such as e.g. chicken embryo fibroblast (CEF) cells), insect cells (such as e.g. D. melanogaster cells, B. mori cells, etc.), bacterial cells (such as e.g. E. coli cells), fungal cells (such as e.g. S. cerevisiae cells), and plant cells (such as e.g. A. thaliana cells). A bioprocess typically results in the production of a product, which can be the cells themselves (e.g. a cell population for use in further bioprocesses, a cell population for use in cell therapy, a cell population for use as a product such as a probiotic, feedstock, etc.), a macromolecule or macromolecular structure such as a protein, peptide, nucleic acid or viral particle (e.g. a monoclonal antibody, immunogenic protein or peptide, a viral or non-viral vector for gene therapy, enzymes such as e.g. for use in the food industry, for environmental applications such as water purification, decontamination, etc.), or a small molecule (e.g. alcohols, sugars, amino acids, etc.).

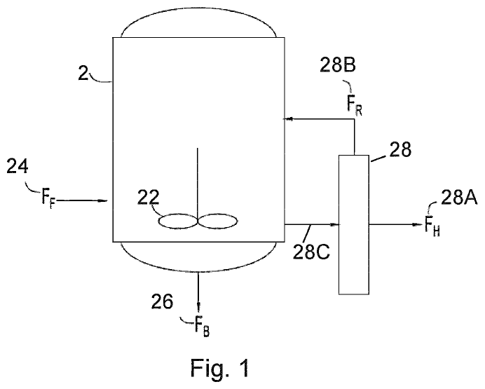

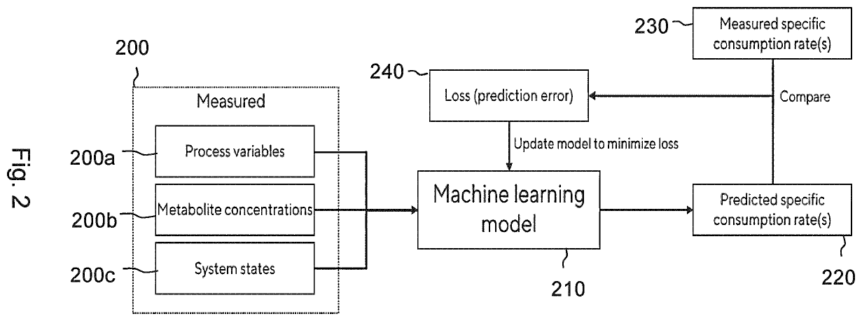

FIG. 1 shows a simplified process diagram for a generic bioprocess. The bioprocess is implemented in the reactor 2, which in the embodiment shown is equipped with agitation means 22. Four flows (also referred to herein as “streams”) are depicted, although depending on the particular situation any or all of these flows may be absent. A first flow 24 is a feed flow F_{F} containing anything that is added to the culture in the bioreactor (typically this includes fresh medium, in which case the bioprocess may be referred to as a “fed-batch” process, “perfusion” process or “continuous” process), a second flow 26 is a bleed flow F_{B} that has the same composition as the culture in the bioreactor, a third flow 28A is a harvest flow F_{H} which is obtained through the processing of an auxiliary harvest stream 28C through a cell separation means 28 to produce the third (harvest) and fourth flow 28B, the fourth flow 28B being a recycle flow F_{R} comprising the cells and any culture medium that has not been completely separated out in the cell separation means 28. In embodiments, the recycle flow F_{R} may be ignored as considering only the harvest flow F_{H} is sufficient to capture the flow that is effectively output from the bioreactor through the harvesting and cell separation process. Therefore, the reference to a harvest flow being present or absent may refer to the auxiliary harvest stream 28C (and derived harvest and recycle flows—F_{H} and F_{R}) being present or absent. This harvest flow F_{H} may be assumed to comprise medium that has the same composition as the medium in the reactor, but no or few cells. The feed, bleed and harvest flows (F_{F}, F_{B}, F_{H} and F_{R}) may all be absent, in which case the bioprocess is referred to as an “unfed batch process” or simply “batch process”. When a feed flow F_{F} and a harvest flow F_{H} are provided, the bioprocess may be referred to as a “perfusion” culture. When a feed flow F_{F} and a bleed flow F_{B} are provided such that the bioprocess is operated at (pseudo) steady-state (from a process condition point of view, i.e. maintaining in particular the volume of the culture constant), the bioprocess may be referred to as a “continuous” culture. When a feed flow F_{F} is provided in the absence of output flows (bleed and harvest flows, F_{B} and F_{H}), the bioprocess may be referred to as a “fed-batch” process. The present invention is applicable to all of the above operating modes.

Products of a bioprocess may have one or more critical quality attributes (CQAs). As used herein, a “critical quality attribute” is any property of a product (including in particular any chemical, physical, biological and microbiological property) that can be defined and measured to characterize the quality of a product. The quality characteristics of a product may be defined to ensure that the safety and efficacy of a product is maintained within predetermined boundaries. CQAs may include in particular the molecular structure of a small molecule or macromolecule (including in particular any of the primary, secondary and tertiary structure of a peptide or protein), the glycosylation profile of a protein or peptide, etc. A product may be associated with a “specification” which provides the values or ranges of values of one or more CQAs, that a product must comply with. A product may be referred to as “in-specification” (or “according to specification”, “within specification”, etc.) if all of its CQAs comply with the specification, and “non-specification” (or “out-of-specification”) otherwise. CQAs may be associated with a set of critical process parameters (CPPs), and ranges of values of the CPPs (optionally maturity-dependent ranges) that lead to acceptable CQAs. A bioprocess run (i.e. a particular instance of execution of a bioprocess) may be referred to as “normal” or “in-specification” if the CPPs are within the predetermined ranges that are believed to lead to acceptable CQAs, and “not normal” or (“out-of-specification”) otherwise.

As used herein, the term “maturity” refers to a measure of completion of a bioprocess. Maturity is commonly captured in terms of time from the start of a bioprocess to the end of the bioprocess. Therefore, the term “maturity” or “bioprocess maturity” may refer to an amount of time from a reference time point (e.g. the start of the bioprocess). As such, the wording “as a function of bioprocess maturity” (e.g. quantifying a variable “as a function of bioprocess maturity”) may in some embodiments refer to “a function of time” (e.g. quantifying a variable “as a function of time, e.g. since the start of the bioprocess”). Conversely, references to a time dependent variable (whether in the text or in equations) should be understood as applicable to any measure of maturity (including but not limited to time), unless the context indicates otherwise. In particular, any measure that increases monotonically as a function of time could be used, such as e.g. the amount of a desired product (or undesired by-product) accumulating in the medium or extracted since the start of a bioprocess, the integrated cell density, etc. may be used. The maturity may be expressed in terms of percentage (or other fractional measure) or as an absolute value that progresses to a value (typically a maximum or minimum value), at which point the bioprocess is considered complete.

As used herein, the term “process condition” refers to any measurable physico-chemical parameter of operation of a bioprocess. Process conditions may include in particular parameters of the culture medium and bioreactor operation, such as e.g. the pH, temperature, medium density, volumetric/mass flowrate of material in/out of the bioreactor, volume of the reactor, agitation rate, etc. Process conditions may also include measurements of the biomass in the bioreactor (e.g. total cell, viable cell density, etc.) or the quantity of a metabolite in a compartment as a whole (including in particular the quantity of a metabolite in any of the cell compartment, culture compartment including culture medium and cells, and the culture medium compartment) of the bioprocess.

As used herein, the term “process output” refers to a value or set of values that quantify the desired outcome of a process. The desired outcome of a process may be the production of biomass itself, the production of one or more metabolites, the degradation of one or more metabolites, or a combination of these.

The term “metabolite” refers to any molecule that is consumed or produced by a cell in a bioprocess. Metabolites include in particular nutrients such as e.g. glucose, amino acids etc., by-products such as e.g. lactate and ammonia, desired products such as e.g. recombinant proteins or peptides, complex molecules that participate in biomass production such as e.g. lipids and nucleic acids, as well as any other molecules such as oxygen (O₂) that are consumed or produced by the cell. As the skilled person understands, depending on the particular situation, the same molecule may be considered a nutrient, a by-product or a desired product, and this may even change as a bioprocess is operated. However, all molecules that take part in cellular metabolism (whether as an input or output of reactions performed by the cellular machinery) are referred to herein as “metabolites”.

Cell Metabolic Condition

The terms “cell metabolic condition” (also referred to herein as “metabolic condition” or “cell condition”) refer to the value of one or more variables that characterize the metabolism of cells in a bioprocess (i.e. metabolic activity of the cells in a bioprocess). This may include in particular the specific transport rate of a metabolite into/out of cells—also referred to herein as the specific consumption rate (e.g. when the metabolite is primarily consumed by the cells, such as a nutrient) or the specific secretion rate (when the metabolite is primarily produced by the cell; this can also be referred to as a specific production rate (SPR), particularly when the metabolite is a desired product), or any variable that is derived from a set of variables that includes one or more of these (e.g. using multivariate analysis techniques as will be described further below). For example, in some embodiments the metabolic condition of a cell culture can be expressed as [metabolic condition]=ƒ(δ, u, m, s) where f is a function that transforms an original set of variables into one or more variables that capture the relationship between the original variables, δ are the specific secretion/consumption rates of one or more metabolites, u are process variables such as temperature, pH, etc., m are metabolite concentrations, and s are variables representing the state of the cell culture system. A state can be a variable that is modeled by a system of differential equations that together form a kinetic growth model, as will be explained further below. For example, state variables of the cell culture system can include one or more of viable cell density, lysed cell density, total cell density, dead cell density, or related variables such as cell viability. Function f can be obtained, for example, using PCA, PLS or OPLS as further explained below. The cell consumption or secretion/production rate of a metabolite (i.e. the specific transport rate of the metabolite into/out of cells) and the concentration of the metabolite inside a cell (which may be expressed in terms of units of mass per volume or per cell) may be considered to represent metabolic variables (in that they characterize the metabolism of the cell). Note that a metabolite may be transported both into and out of cells (e.g. a metabolite may be both consumed and produced), in which case the specific transport rate quantifies the combined effect of the movement in both directions. In other words, the specific transport rate of a metabolite quantifies the net amount of metabolite transported between the cells and the liquid medium (e.g. as a change in the amount of metabolite in the medium, reflecting both movement from the medium to the cells and vice versa). Further, the concentration of the same metabolite in a compartment of the bioprocess (e.g. in the bulk composition or the liquid medium, which may be expressed in terms of units of mass per volume) may be considered to represent a process variable (in that it characterizes a macroscopic process variable). For example, the concentration of oxygen or glucose in the liquid medium (e.g. in mass/volume) may be considered to be a process variable (also referred to herein as “process parameter”), describing the process at a macroscopic level (process condition), whereas the concentration of oxygen or glucose in the cells (e.g. in mass/cell) may be considered to be a metabolic variable, describing the metabolic condition of the cells.

The term “multivariate statistical model” refers to a mathematical model that aims to capture the relationships between multiple variables. Common multivariate statistical models include principal components analysis (PCA), partial least square regression (PLS) and orthogonal PLS (OPLS). The term “multivariate statistical analysis” refers to the building (including but not limited to the design and parameterisation) and/or using of a multivariate statistical model.

Principal component analysis (PCA) is used to identify a set of orthogonal axes (referred to as “principal components”) that capture progressively smaller amounts of the variance in the data. The first principal component (PC1) is the direction (axis) that maximizes the variance of the projection of a set of data onto the PC1 axis. The second principal component (PC2) is the direction (axis) that is orthogonal to PC1 and that maximizes the variance of the projection of the data onto the PC1 and PC2 axes. The coordinates of a data point in the new space defined by one or more principal components is sometimes referred to as “scores”. PCA functions as a dimensionality reduction approach, resulting in scores for each data point that capture the contribution of multiple underlying variables to the diversity in the data. PCA can be used on historical data about a set of runs of a bioprocess, to characterize and distinguish good (normal) and bad (not normal) process conditions. This enables the retrospective identification of when a historical batch has deviated outside of acceptable process conditions, and to interpret which of the individual process variables are most responsible for the deviation observed in the global process condition. This can then be used to investigate how to avoid such a deviation in the future.

PLS is a regression tool that identifies a linear regression model by projecting a set of predicted variables and corresponding observable variables onto a new space. In other words, PLS identifies the relationship between a matrix of predictors X (dimension m×n) and a matrix of responses Y (dimension m×p) as:

X=TP^{t}+E (1)

Y=UQ^{t}+F (2)

where T and U are matrices of dimension m×l that are, respectively, the X score (projections of X onto a new space of “latent variables”) and the Y scores (projections of Y onto a new space); P and Q are orthogonal loading matrices (that define the new spaces and respectively have dimensions n×l and p×l); and matrices E and F are error terms (both assumed to be IID—independent and identically distributed—random normal variables). The scores matrix T summarizes the variation in the predictor variables in X, and the scores matrix U summarizes variation in the responses in Y. The matrix P expresses the correlation between X and U, and the matrix Q expresses the correlation between Y and T. The decomposition of X and Y into a matrix of scores and corresponding loadings is performed so as to maximize the covariance between T and U. OPLS is a variant of PLS where the variation in X is separated into three parts: a predictive part that is correlated to Y (TP^{t} as in the PLS model), an orthogonal part (TₒᵣₜₕPₒᵣₜₕ^{t} which captures systematic variability that is not correlated to Y) and a noise part (E as in the PLS model—which captures residual variation). Partial least squares (PLS) and orthogonal PLS (OPLS) regression can be used to characterize the impact that the process condition has on a desired process output (concentration of product, quality attribute, etc.). This can be performed by fitting an (O)PLS model as described above, with X including the one or more process variables that are believed to have an effect on the process output, and Y including the corresponding measures of process output. This can be used to identify which process variables can be manipulated, and how they should be manipulated, to improve or control a desired output.

The Umetrics® software suite (Sartorius Stedim Data Analytics) further includes so called “batch evolution models” (BEM) which describe the time-series evolution of process conditions, referred to as the process ‘path’. The process paths are obtained by fitting an (O)PLS model as described above, but where X includes the one or more process variables that are believed to be of potential relevance, measured at multiple times (maturity values) over the evolution of the process, and Y includes the corresponding maturity values. For example, a set of n process variables may have been measured at m maturity values, and these n×m values may be included as a coefficient in the matrix X. The corresponding matrix Y is an m×1 matrix (i.e. a vector of length m) of maturity values. The T matrix therefore comprises scores values for each of the m maturity values and each of l identified latent variables that describe the aspects of the process variables that are most correlated with maturity. By training the BEM on process paths that resulted in the desired product quality at the end of the process, a “golden BEM” can be defined that describes the range of process paths that are acceptable for a future batch (leading to CQAs within specifications), using the values of the scores in T. This enables the monitoring of batches to know that an ongoing batch is within specification. It also means that if an ongoing batch looks like it will deviate from the accepted range of paths, then an alarm can be raised to the operator to let them know that corrective action needs to be taken to prevent loss of product. Furthermore, the process measurements that are contributing to the deviation in process condition can be highlighted to the operator (by analyzing the variables in X that most contribute to the score in T that has been observed to deviate from the expected course) to assist in diagnosing the problem and identifying the appropriate course of corrective action. This can all be done in real-time. Furthermore, operators only need to consider a small set of summary parameters during normal batch operation with the option to drill-down into specifics with an appropriate subject matter expert only when something is going wrong.

Calculation of Specific Transport Rates Using Material Balance Equations

The specific transport rate (i.e. specific consumption or production/secretion rate, depending on whether the rate has a positive or a negative sign as seen from the perspective of the reactor) of metabolite i (denoted δ_{m,i} below) is an important variable that captures aspects of the metabolic condition of the cells in the bioprocess. Further, where the metabolite is a desired product, its specific secretion/production rate provides a useful indication of the productivity of the cell culture. These variables δ_{m,i} (one for each metabolite of interest) can be calculated at a particular time point using material balance equations as will be described below, together with measurements of the concentration of the respective metabolite and the viable cell density at two successive time points.

Equations that represent the evolution of the bulk concentration of metabolites (including in particular nutrients, by-products and the desired product) can be expressed based on a material balance equation such as equation (3) below:

[total change of metabolite amount in reactor]=[total flow of metabolite into reactor]−[total flow of metabolite out of reactor]+[secretion of metabolite by cells in reactor]−[consumption of metabolite by cells in reactor] (3)

Equation (3) expresses in mathematical form the conservation of mass in the system (reactor), for a metabolite under investigation. At every time point t, equation (3) must be satisfied. The flows of metabolite in equation (3) may be expressed as mass flows or molar flows (as the latter can be converted to the former and vice-versa using molar mass, such that the conservation of mass expressed in the equation is verified regardless of the units chosen), and the skilled person would be able to convert one into the other. As such, references to mass flows are intended to encompass the use of the corresponding molar flows with corresponding adjustments for consistency of units within an equation. Similarly, references to concentrations may refer to the mass or molar concentrations. The flow of the metabolite into the bioreactor depends on the value of the feed flow F_{F} and the concentration of the metabolite in this flow (if this flow is present, i.e. F_{F}≠0). The flow of the metabolite out of the bioreactor depends on the value of the harvest flow F_{H} (if present) and the value of the bleed flow F_{B} (if present), and the concentration of the metabolite in these respective flows. The consumption and secretion of a metabolite by the cells in the bioreactor depends on the viable cell density in the reactor and on a variable called the “specific transport rate” (sometimes also referred to as “metabolic rate”), which can also be referred to as “specific consumption rate” (typically, if it is negative, i.e. the metabolite is being consumed by the cells) or the “specific secretion/production rate” (typically, if it is positive, i.e. the metabolite is being produced by the cells) of the metabolite by the cells. Therefore, the material balance described in equation (3) can be written for a general system (e.g. as illustrated on FIG. 1), for metabolite i as equation (4) below:

$$\frac{d ( {m}_{i} )}{dt} = \frac{{F}_{F}}{V} * {m}_{F , i} - \frac{{F}_{H}}{V} * {m}_{H , i} - \frac{{F}_{B}}{V} * {m}_{B , i} + {δ}_{m , i} * {x}_{v} \qquad ( 4 )$$

where δ_{m,i} is the specific transport rate of metabolite i by the cells in the culture, mᵢ is the concentration of metabolite i in the reactor, V is the volume of the culture in the bioreactor, m_{F,i} is the concentration of metabolite i in the feed flow, m_{H,i} is the concentration of metabolite i in the harvest flow, m_{B,i} is the concentration of metabolite i in the bleed flow, xᵥ is the viable cell density in the reactor, and F_{F}, F_{H} and F_{B} are the volumetric feed, harvest and bleed flow rates, respectively (although mass flow rates can equally be used with the appropriate coefficients for densities of the respective flows). In embodiments, the term δ_{m,i}*xᵥ can be replaced by a term

$${δ}_{m , i} * {x}_{v} * ( \frac{{m}_{i}}{{m}_{i} + ε} )$$

where ε is a constant that is chosen to ensure that the values of mᵢ that are below the detection limit for the metabolite do not cause errors in the estimation of specific transport rates. Typically, ε is chosen to be approximately equal to the detection limit for the metabolite (e.g. it can be chosen as 0.05 where the metabolite concentration is standardized).

Equation (4) assumes that the harvest flow 28A contains the only material leaving the system through the auxiliary harvest flow 28C (i.e. the auxiliary harvest flow and the return flow do not need to be included in the model as the metabolite only leaves the system through the harvest flow), and that the cell separation device 28 functions such that the harvest flow 28A can be assumed to contain no cells. Equation (4) can be adapted to include an auxiliary harvest flow 28C (and corresponding m_{A,i} and density ρ_{A} if a mass flow rate is used) and a return flow 28B (and corresponding m_{R,i} and ρ_{R}). Further, equation (4) can be amended to model the removal of some cells through the harvest flow. In other words, additional terms can be added to equation (4) and some can be removed depending on the set-up of the bioprocess and the assumptions made. Examples of a few common bioprocess set ups and their corresponding simplified equations are provided below.

As the skilled person understand, the general equation in (3) can be expressed differently depending on the operating mode (e.g. fed-batch, unfed-batch, etc.) and the assumptions made (e.g. variable volume, variable concentration in the various flows and in the bioreactor, etc.). The skilled person would be able to express and solve equation (3) accordingly, in light of the teaching provided herein. Further, whether a particular assumption is reasonable may depend on the situation, and the skilled person would be able to verify whether this is the case using well known techniques. For example, the skilled person would be able to verify whether the volume of a culture is constant (e.g. by examining the amounts of material flowing in and out of the bioreactor or using a level sensor), whether the medium density is constant (e.g. using a hydrometer), whether the concentration of one or more metabolites is the same in one or more compartments and/or flows (e.g. using one or more metabolite sensors to measure metabolite concentration separately in these compartments and/or flows), etc. The skilled person would further be aware that a particular assumption may be reasonable in one case but not in another. For example, the concentration of a small molecule metabolite in the culture medium may be likely to be the same in the bioreactor and in the out flows (harvest and/or bleed flows), whereas the concentration of a macromolecule may differ between the bioreactor and one or more of the out flows if the macromolecule is likely to be held up on filters or other structures.

The specific transport (consumption/secretion) rate of a metabolite at a particular time point can be calculated based on equation (4) (or its simplified variants as explained below), using known (i.e. measured or simulated) values of the metabolite concentration and the viable cell density, and using a first order finite difference approximation. For example, using such an approximation, equation (4) can be solved for δ_{m,i} at time k (denoted δ_{m,i}(tₖ) or δ_{m,i,k}) according to equation (5):

$${δ}_{m , i} ( {t}_{k} ) = \frac{1}{IVC {D}_{k} {V}_{k}} [ \text{} \frac{( {F}_{F , k + 1} + {F}_{F , k} )}{2} {m}_{F , i} ( {t}_{k + 1} - {t}_{k} ) - \frac{( {F}_{B , k + 1} + {F}_{B , k} )}{2} {m}_{B , i} ( {t}_{k + 1} - {t}_{k} ) - \frac{( {F}_{H , k + 1} + {F}_{H , k} )}{2} {m}_{H , i} ( {t}_{k + 1} - {t}_{k} ) - {V}_{k} ( {m}_{i , k + 1} - {m}_{i , k + 1} ) ] \qquad ( 5 )$$

where the subscripts k and k+1 indicate a value at the k^{th} and k+1^{th} time points where values for the metabolite concentration and viable cell density are available, and IVCDₖ is the integrated viable cell density between the time points k and k+1. Note that the consumption rate at the k^{th} observation is forward looking, meaning that it represents the consumption rate for the time interval k→k+1.

The specific transport (consumption/secretion) rates of one or more metabolites of interest can be used as variables of a metabolic condition model that classifies the metabolic condition of the cells e.g., into an optimal or suboptimal state or category for biomaterial production, as will be explained further below.

For a perfusion culture (where a feed flow F_{F}, a bleed flow F_{B} and a harvest flow F_{H} are present), equation (4) can be simplified by making a few assumptions. For example, assuming that the metabolite concentration is the same everywhere in the culture medium in the bioreactor, and hence also in the harvest flow and in the bleed flow (in other words, assuming that concentration gradients within the reactor are negligible such that m_{B,i}=m_{H,i}=mᵢ), and that the number of cells lost in the bleed and harvest flows is negligible, equation (4) can be written as:

$$\frac{d ( {m}_{i} )}{dt} = \frac{{F}_{F}}{V} * {m}_{F , i} - \frac{( {F}_{H} + {F}_{B} )}{V} * {m}_{i} + {δ}_{m , i} * ( {x}_{v} ) \qquad ( 4 a )$$

Assuming further that the volume of the culture is constant (i.e. F_{F}=F_{H}+F_{B}), that the flows are constant, and using a first-order finite difference approximation for the derivative, equation (18a) can be resolved for the metabolite specific consumption/secretion rate at time tₖ as:

$${δ}_{m , i} ( {t}_{k} ) = \frac{\begin{matrix}V * ( {m}_{i , k + 1} - {m}_{i , k} ) - {F}_{F} * {m}_{F , i , k} * ( {t}_{k + 1} - {t}_{k} ) + \\ {F}_{F} * {m}_{i , k} * ( {t}_{k + 1} - {t}_{k} )\end{matrix}}{V * {IVCD}_{k}} . \qquad ( 5 a )$$

For a fed-batch culture (where a feed flow is present, but no bleed flow or harvest flow are present, i.e. F_{H}=F_{B}=0), equation (18) can be written as:.

$$\frac{d ( {m}_{i} )}{dt} = \frac{{F}_{F}}{V} * {m}_{F , i} + {δ}_{m , i} * ( {x}_{v} ) \qquad ( 4 b )$$

Using a first-order finite difference approximation for the derivative, equation (4b) can be resolved for the metabolite specific consumption/secretion rate at time tₖ as:

$${δ}_{m , i} ( {t}_{k} ) = \frac{{F}_{F} * {m}_{F , i , k} * ( {t}_{k + 1} - {t}_{k} ) - ( ( {V}_{k + 1} + {V}_{k} ) / 2 ) * ( {m}_{i , k + 1} - {m}_{i , , k} )}{( ( {V}_{k + 1} + {V}_{k} ) / 2 ) * {IVCD}_{k}} . \qquad ( 5 b )$$

The approach in equation (5b) may be particularly useful in embodiments where the feed-flow is continuous or semi-continuous, such as e.g. in drip feed flows. In embodiments where a bolus feed strategy is implemented (i.e. the feed flow is provided as relatively large instantaneous additions), equation (4b) can be rewritten using a pseudo metabolite concentration pmᵢ, that allows the feed flow to be eliminated from equation (4b), i.e.:

$$\frac{d ( {pm}_{i} )}{dt} = {δ}_{m , i} * ( {x}_{v} ) . \qquad ( 4 d )$$

For metabolites that are provided in the feed flow, a pseudo metabolite concentration pmᵢ may be obtained by: (i) using the measured (or otherwise determined, such as e.g. based on the initial reactor volume and the volumes provided in the one or more feed bolus) reactor volume and known feed concentrations to determine how much of the metabolite is added to the reactor with each feed, and (ii) subtracting the value in (i) from all measurements of the metabolite's concentration after the feed. For metabolites that are not present in the feed (or that can be assumed not to be present in the feed), a pseudo metabolite concentration pmᵢ may be obtained by: (i) using the measured (or otherwise determined, such as e.g. based on the initial reactor volume and the volumes provided in the one or more feed bolus) reactor volume to determine the change in concentration due to dilution that is caused by each feed, and (ii) adding the value in (i) from all measurements of the metabolite's concentration after the feed. Equation (4d) can be resolved for the metabolite specific transport rate at time k using a first-order finite difference approximation for the derivative:

$${δ}_{m , i} = \frac{\frac{dpmi}{dt}}{{x}_{v , k}} = \frac{{pm}_{i , k + 1} - {pm}_{i , k}}{{IVCD}_{k}} . \qquad ( 5 d )$$

Equation (5d) can also be written as:

$${δ}_{m , i} ( {t}_{k} ) = \frac{( {m}_{i , k + 1} - {m}_{i , k} - {mAdd}_{i , k} / {V}_{k} )}{{IVCD}_{k}} \qquad ( 5 e )$$

where m_{i,k} is the concentration of metabolite i at time k, mAdd_{i,k} is the amount of metabolite i in the bolus addition of metabolite i at time k, Vₖ is the total volume in the bioreactor, and iVCD is the integrated Viable Cell Density. Further, where the metabolite is a product of the cells (i.e. a metabolite that is not expected to be present in the feed, such as e.g. a desired product), the specific production rate of the metabolite can be written as:

$${δ}_{m , i} ( {t}_{k} ) = \frac{( {m}_{i , k + 1} - {m}_{i , k} )}{{IVCD}_{k}} \qquad ( 5 f )$$

where δ_{m,i}(tₖ) can also be written as q_{IgG}(tₖ) and m_{i,k+1}, m_{i,k} can also be written as C_{IgG,k+1}, C_{IgG,k} to represent that the metabolite is a desired product such as e.g. a recombinant antibody (IgG).

For an unfed-batch culture (where no feed flow, bleed flow or harvest flow is present, i.e. F_{F}=F_{H}=F_{B}=0), equation (4) can be written as:

$$\frac{d ( {m}_{i} )}{dt} = {δ}_{m , i} * ( {x}_{v} ) \qquad ( 4 c )$$

which can be resolved for the specific consumption/secretion rate at time k as:

$${δ}_{m , i , k} = \frac{\frac{dmi}{dt}}{{x}_{v , k}} = \frac{{m}_{i , k + 1} - {m}_{i , k}}{{IVCD}_{k}} . \qquad ( 5 c )$$

In embodiments, IVCDₖ may be calculated using equation (6):

IVCDₖ=(α x_{v,k}+β x_{v,k+1})*(tₖ₊₁−tₖ) (6)

where the coefficients α and β weight the relative influence of the two viable cell density values and are such that α+β=1. For example, the two values may be weighted identically, i.e. α=β=0.5. In embodiments, α and β are chosen such that α>β (for example α=0.6 and β=0.4). These coefficients may be chosen to reflect the observed cell growth behaviour, and may be chosen independently for each time point. Where exponential growth can be assumed, the integrated viable cell density can be calculated using a log transform. For example, the integral cell density may be calculated using equation (6a).

$${IVCD}_{k} = {x}_{v , k} * {e}^{{u}_{k} ( {t}_{k + 1} - {t}_{k} )} \qquad ( 6 a )$$

$$where {μ}_{k} = \frac{ln {(}^{{x}_{v , k + 1}} {/}_{{x}_{v , k}} )}{{t}_{k + 1} - {t}_{k}} \qquad ( 6 b )$$

Alternatively, any methods to calculate an integrated viable cell density may be used in the methods described herein.

The equations for δ_{m,i} at time k as described above (or any corresponding equation that may be defined in view of the configuration of the process and the set of assumptions made) can be solved using the known (typically measured) biomass and metabolite concentrations at times/maturities k and k+1 to obtain a metabolite transport rate at every time point/maturity value where the above measurements are available. Further, this can be performed individually for each measured metabolite. The resulting metabolite transport rates characterize the metabolic condition of the cells in the culture as a function of maturity, and are expressed as an amount of metabolite (mass or moles) per cell per unit of maturity (i.e. typically per unit of time). This represents very valuable information regarding the metabolic condition of the cells, which information can be used by a metabolic condition model to monitor the cell culture as will be described further below. Note that in all equations for specific transport rates, the signs of all the terms can be inverted depending on whether a negative rate is taken to mean that the cells consume the metabolite and a positive rate is taken to mean that the cells produce the metabolite (i.e. the rate is seen from the perspective of the culture medium), or the opposite (i.e. a positive rate means that the cells consume the metabolite and a negative rate means that the cells produce the metabolite, in other words the rate is seen from the perspective of the cells compartment).

Prediction of Specific Transport Rates Using Machine Learning

Using the above approach, specific transport rates may only be accurately calculated at time points where metabolite concentration and viable cell density data is available. This limits our ability to predict the behaviour of a bioprocess, whether for the purpose of predictive control, process optimization or simulation (digital twin). This is addressed by the present invention, which uses machine learning approaches (i.e. machine learning algorithms that train and/or deploy particular machine learning models) to predict the specific transport rates of one or more metabolites at a future time point, based on the known (measured or calculated) values of one or more variables that characterize the bioprocess at one or more previous time point.

In other words, according to the present invention, the specific consumption/secretion rate of a metabolite is obtained as δᵢ=f_{ML,i}(u, m, s) where f_{ML,i} is a model that has been trained to predict the specific consumption/secretion rate δᵢ (alone or jointly with other δᵢ) as a function of one or more variables selected from process variables u (e.g. temperature, pH, etc.), one or more metabolite concentrations m (where metabolite concentrations can also be considered to form a part of the process variables, i.e. the notation u can refer to physico-chemical process variables and/or to metabolite concentrations in the bulk medium), and one or more variables s representing the state of the cell culture system (e.g. viable cell density, lysed cell density, total cell density, cell viability). For the avoidance of any doubt, any of the categories of variables may be absent, i.e. the model f_{ML,i}(u, m, s) may comprise no variables selected from process variables u, no variables selected from metabolite concentrations m, and/or no variables selected from cell culture state variables s (provided that at least one variable of at least one of these categories is included). In embodiments, the one or more variables comprise at least a metabolite concentration m. Thus, in some embodiments, the process variables and/or the cell state variables may be absent, and the trained model may predict the specific consumption/secretion rate δᵢ based at least (or solely) on one or more metabolite concentrations. Preferably, the one or more metabolite concentrations include the concentration of the metabolite for which a specific consumption rate is being predicted, and/or a metabolite whose concentration is highly correlated with that of the metabolite whose specific consumption rate is being predicted (such as e.g. an immediate product or precursor thereof). Without wishing to be bound by theory, it is believed that a machine learning that performs satisfactorily at the task of predicting a specific transport rate may be obtained using only metabolite concentrations as these are naturally correlated to the specific transport rates. However, process and cell culture state variables may carry complementary information, and a machine learning model may advantageously learn to use this information to improve its prediction accuracy. Thus, including more and/or a greater variety of predictive variables (such as e.g. including variables from one or more or each of the u, m and s categories) may result in a model that has improved predictive accuracy and/or that is able to achieve good and/or improved level of accuracy in a greater variety of conditions.

The term “machine learning model” refers to a mathematical model that has been trained to predict one or more output values based on input data, where training refers to the process of learning, using training data, the parameters of the mathematical model that result in a model that can predict outputs values with minimal error compared to comparative (known) values associated with the training data (where these comparative values are commonly referred to as “labels”). The term “machine learning algorithm” or “machine learning method” refers to an algorithm or method that trains and/or deploys a machine learning model. The machine learning models used in the present invention can be seen as regression models in that they capture the relationship between a dependent variable (the specific transport rates that are being predicted) and a set of independent variables (also referred to as predictors). Any machine learning regression model may be used according to the present invention. In the context of the present invention, a machine learning model is trained by using a learning algorithm to identify a function F: u, m, s→δᵢ where F is a function parameterized by a set of parameters θ such that:

δᵢ≈\hat{δ}ᵢ=F(u, m, s|θ) (7)

where \hat{δ}ᵢ is the predicted specific transport (consumption/secretion) rate and θ is a set of parameters identified as satisfying equation (8):

θ=argmin_{θ}L(δᵢ, \hat{δ}ᵢ) (8)

where L is a loss function that quantifies the model prediction error based on the observed and predicted specific consumption rates. The specific choice of the function F, parameters θ and function L as well as the specific algorithm used to find θ (learning algorithm) depends on the specific machine learning method used. Any method that satisfies the equations above can be used within the context of the present invention, including in particular any choice of loss function, model type and architecture. In embodiments, a machine learning model is a linear regression model. A linear regression model is a model of the form according to equation (9), which can also be written according to equation (9b):

Y=Xβ+ε (9)

yᵢ=β₀+β₁xᵢ₁+ . . . βₚxᵢₚ+εᵢ i=1, . . . , n (9b)

where Y is a vector with n elements yi (one for each dependent variable), X is a matrix with elements xᵢ₁ . . . xᵢₚ for each of the p predictor variables and each of the n dependent variables, and n elements of 1 for the intercept value, β is a vector of p+1 parameters, and ε is a vector of n error terms (one for each of the dependent variables).

In embodiments, a machine learning model is a random forest regressor. Random forest regressors are described in e.g. Breiman, Leo. “Random forests.” Machine learning 45.1 (2001): 5-32. A random forest regressor is a model that comprises an ensemble of decision trees and outputs a class that is the average prediction of the individual trees. Decision trees perform recursive partitioning of a feature space until each leaf (final partition sets) is associated with a single value of the target. Regression trees have leaves (predicted outcomes) that can be considered to form a set of continuous numbers. Random forest regressors are typically parameterized by finding an ensemble of shallow decision trees. In embodiments, a machine learning model is an artificial neural network (ANN, also referred to simply as “neural network” (NN)). ANNs are typically parameterized by a set of weights that are applied to the inputs of each of a plurality of connected neurons in order to obtain a weighted sum that is fed to an activation function to produce the neuron's output. The parameters of an NN can be trained using a method called backpropagation (see e.g. Rumelhart, David E., Geoffrey E. Hinton, and Ronald J. Williams. “Learning representations by back-propagating errors.” Nature 323.6088 (1986): 533-536) through which connection weights are adjusted to compensate for errors found in the learning process, in combination with a weight updating procedure such as stochastic gradient descent (see e.g. Kiefer, Jack, and Jacob Wolfowitz. “Stochastic estimation of the maximum of a regression function.” The Annals of Mathematical Statistics 23.3 (1952): 462-466).

Suitable loss functions for use in regression problems such as those described herein include the mean squared error, the mean absolute error and the Huber loss. Any of these can be used according to the present invention. The mean squared error (MSE) can be expressed as:

L(⋅)=MSE(δᵢ, \hat{δ}ᵢ)=(δᵢ−\hat{δ}ᵢ)² (10)

The mean absolute error (MAE) can be expressed as:

L(⋅)=MAE(δᵢ, \hat{δ}ᵢ)=|δᵢ−\hat{δ}ᵢ| (11)

The MAE is believed to be more robust to outlier observations than the MSE. The Huber loss (see e.g. Huber, Peter J. “Robust estimation of a location parameter.” Breakthroughs in statistics. Springer, New York, N.Y., 1992. 492-518) can be expressed as:

$$L ( {δ}_{i} , \hat{{δ}_{ι}} \vert a ) = \{ \begin{matrix}0.5 {( {δ}_{i} - {\hat{δ}}_{i} )}^{2} for \vert {δ}_{i} - {\hat{δ}}_{i} \vert < a \\ a \vert {δ}_{i} - {\hat{δ}}_{i} \vert - 0. 5 {a}^{2} otherwise\end{matrix} \qquad ( 12 )$$

where α is a parameter. The Huber loss is believed to be more robust to outliers than MSE, and strongly convex in the neighborhood of its minimum. However, MSE remains a very commonly used loss functions especially when a strong effect form outliers is not expected, as it can make optimization problems simpler to solve.

In embodiments, a machine learning model comprises an ensemble of models whose predictions are combined. Alternatively, a machine learning model may comprise a single model. In embodiments, a machine learning model may be trained to predict a specific secretion/consumption rates for a single metabolite. Alternatively, a machine learning model may be trained to jointly predict a specific secretion/consumption rates for a plurality of metabolites. In such cases, the loss function used may be modified to be an (optionally weighted) average across all variables that are predicted, as described in equation (13):

$${L}_{M} ( δ , \hat{δ} ) = \frac{1}{n} {\sum }_{i ∈ m} {α}_{i} L ( {δ}_{i} , {\hat{δ}}_{i} ) \qquad ( 13 )$$

where αᵢ are optional weights that may be individually selected for each of the metabolites i, δ and \hat{δ} are the vectors of actual and predicted specific consumption rates of all metabolites. Optionally, the values of δᵢ may be scaled prior to inclusion in the loss function (e.g. by normalizing so that the labels for all the jointly predicted variables have equal variance), for example to reduce the risk of some of the jointly predicted \hat{δ}ᵢ dominating the training.

In embodiments, the machine learning model is trained to predict a specific transport rate for one or more metabolites, based on input values comprising the known (measured or calculated) values of one or more variables that characterize the bioprocess at a time point k. In embodiments, the machine learning model is trained to predict a specific transport rate for one or more metabolites, based on input values comprising the known (measured or calculated) values of one or more variables that characterize the bioprocess at a plurality of time points (k, k−1, . . . ). For example, the machine learning model may be trained to predict a specific transport rate for one or more metabolites, based on input values comprising the known (measured or calculated) values of one or more variables that characterize the bioprocess at a first time point k and a second time point k−1. The input variables may comprise values of one or more variables at each of a plurality of time points, where the one or more variables may be chosen independently for each time point. For example, the one or more variables at one time point may partially or fully overlap with the one or more variables at another time point. In embodiments, the one or more variables are the same for each of the plurality of time points. In some embodiments, missing values may be present in the training data (the data used to train the model) or in the data provided to the model in use. Training and/or using the machine learning model may comprise imputing one or more missing values. Imputation methods are known in the art. Imputation methods suitable for use in the present context include e.g. linear interpolation, mean substitution, etc.

In embodiments, the machine learning model is trained to predict a specific transport rate for one or more metabolites at a future time, based on input values comprising the known (measured or calculated) values of one or more variables that characterize the bioprocess at one or more time points k, k−1, etc. In other words, the training data that is used may be such that the model predictions based on data at one or more time points k, k−1, etc. are evaluated against known corresponding values at a time j>k, k−1, . . . In embodiments, the plurality of time points in the input data used for training are separated by a predetermined time period (e.g. 1 hour, 2 hours, 3 hours, 12 hours, 1 day, 2 days, etc.). For example, the input data used for training may comprise values at one time point and at a second time point separated from the first time point by a fixed period. In embodiments, the model predictions based on data at one or more time points k, k−1, etc. are evaluated against known corresponding values (label values) at a time j>k, k−1, . . . where the time point j is separated from one or more of the time points k, k−1, . . . by a predetermined time period. In a simple example, the machine learning model is trained to predict a specific transport rate for one or more metabolites on the next day (i.e. in 1 day) from the day of the latest one of the input values. In this example, if the input values comprise two time points separated by a fixed period of one day, then the machine learning model is trained to predict a specific transport rate for one or more metabolites on the next day based on the values of one or more variables on the present day and the preceding day.

The time periods (whether between input values or between input values and predicted values) may be approximately the same for the whole training data set. Alternatively, the training data may comprise sets of input values and/or input values and corresponding known (label) values that are not separated by the same time difference. For example, the training data may comprise measurements for a plurality of bioprocesses, where in some of the plurality of bioprocesses data was acquired every day, whereas in others data was acquired every half day. Preferably, the training data used comprises sets of input values and input values and corresponding label values that are separated by approximately the same time (or maturity, as the case may be) differences. In the example above, this can be achieved by only using measurements associated with consecutive days for the training data that includes more than daily measurements, or conversely by imputing measurements for time points in which measurements were not acquired. The trained machine learning model may advantageously be used to predict specific transport rates associated with time differences between prediction and latest input value and/or between multiple input values that are similar to the corresponding time differences in the training data. Reference to times and time differences may refer to corresponding maturities and maturity differences. Further, a machine learning model that has been trained to predict specific transport rates based on input values comprising values at a plurality of time points may be used to predict specific transport rates based on input values comprising missing values (e.g. comprising fewer time points than the number of time points that the machine learning model was trained on). This may be the case for example when values for a single time point are available (e.g. when the machine learning model is used to make a prediction based on initial conditions). Thus, for example, a machine learning model trained to predict specific transport rates based on input values comprising values at two consecutive time points may be used to predict specific transport rates based on input values comprising value at one time point and zero or imputed values for the other of the two consecutive time points that the machine learning model expects as input. In general, missing data may be imputed using a variety of approaches such as replacing a missing value by the mean, median or mode of a corresponding set of values, a value drawn randomly from a corresponding set of values, etc. Alternatively, machine learning algorithms that support missing values may be used. Such algorithms may include k-nearest neighbours or classification and regression trees. For example, the machine learning model may be a random forest.

Kinetic Growth Model

The term “kinetic growth model” refers to any model that captures the kinetic of a cell population in a bioprocess. Accordingly, a kinetic growth model may be used to monitor or simulate the number of live cells (and other culture-related parameters) in a bioreactor and to make predictions about the number of cells in the bioreactor at a future point in time. For example, a kinetic growth model may include one or more differential equations that model the maturity-dependent (typically, time-dependent) behaviour of one or more cell population variables. Cell population variables are a specific type of process conditions that characterize the viable, dead, lysed and/or total cell population in a bioprocess. Typical cell population variables include the viable cell density (VCD), dead cell density, and lysed cell density, which respectively capture the concentration of viable, dead and lysed cells in a bioreactor. In embodiments, a kinetic growth model uses the Monod equation to capture cell growth rates as a function of the concentration of a limiting nutrient. One example of a kinetic growth model is provided in equations (14) to (17) below, which describe respectively changes in the viable cell density xᵥ, the dead cell density x_{d}, the total cell density xₜ and the lysed cell density xₗ as a function of time/maturity:

$$\frac{{dx}_{v}}{dt} = ( {μ}_{eff} - {μ}_{d} - \frac{{F}_{b}}{V} ) {x}_{v} \qquad ( 14 )$$

$$\frac{{dx}_{d}}{dt} = {μ}_{d} {x}_{v} - ( {k}_{l} + \frac{{F}_{b}}{V} ) {x}_{d} \qquad ( 15 )$$

$$\frac{{dx}_{l}}{dt} = {k}_{l} {x}_{d} - \frac{{F}_{h} + {F}_{b}}{V} {x}_{l} \qquad ( 16 )$$

$${x}_{t} = {x}_{v} + {x}_{d} + {x}_{l} \qquad ( 17 )$$

In equations (14)-(17), F_{b} and Fₕare the bleed and harvest rates, respectively (see above and FIG. 1), V is the reactor volume, μ_{eff}, μ_{d}, and kₗ are the effective growth, effective death, and lysing rates, respectively. Equation (14) captures the following assumptions: (i) live cells are formed from live cells at the effective growth rate μ_{eff}; (ii) live cells can exit the reactor through the bleed stream F_{b} (if present); (iii) live cells can be transformed into dead cells at the effective death rate μ_{d}; and (iv) no live cells exit the reactor through the harvest stream Fₕ(in other words, where a harvest stream is present it includes a perfect cell retention filter—which may be a valid assumption when any cells exiting the bioreactor through the harvest stream represent a negligible amount). The calculation of the effective growth rate is critical to the functioning of this model and is presented in detail below.

Equation (15) captures the following assumptions: (i) dead cells are formed from live cells at the effective death rate μ_{d}; (ii) dead cells are converted into lysed cells through a first-order process with rate kₗ; (iii) dead cells can exit the reactor through the bleed stream F_{b} (if present); and (iv) no dead cells exit the reactor through the harvest stream Fₕ. The calculation of the effective death rate is presented in detail below. Equation (16) captures the following assumptions: (i) lysed cells are formed from dead cells at rate kₗ; (ii) lysed cells can exit the reactor through the bleed stream F_{b} (if present); and (iv) lysed cells can exit the reactor through the harvest stream Fₕ (if present). Equation (17) captures the assumption that cells are either viable, dead or lysed. This can be used to calculate one of the variables from the others, for example lysed cells (which typically cannot be measured directly), by closing the balance on living and dead cells.

In embodiments, the death process (captured through the parameter μ_{d}) can be modeled as resulting from a combination of a base death rate and a toxicity factor, for example as provided in equation (18) below.

μ_{d}=k_{d}+kₜϕₜ (18)

In equation (18), k_{d} is the primary (base) death rate, kₜ is the “toxicity rate” and ϕₜ is a measure of toxicity. The variable ϕₜ may represent the concentration of one or more components that have a toxic effect on cells. In embodiments, ϕₜ is assumed to be equal to the concentration of lysed cells xₗ (i.e. ϕₜ=xₗ). In other embodiments, ϕₜ is assumed to be equal to the concentration of an unknown biomaterial that accumulates in the culture as a result of cell growth and inhibits cells growth and/or is toxic to the cells, which is designated as ϕ_{b} (such that ϕₜ=ϕ_{b}). The evolution of this variable can be captured for example using equation (19) below:

$$\frac{d {ϕ}_{b}}{dt} = {x}_{v} - \frac{{F}_{h} + {F}_{b}}{V} {ϕ}_{b} \qquad ( 19 )$$

which assumes that the unknown product is produced at a rate that is proportional to the viable cell density xᵥ and that the product can exit the bioreactor through the bleed and harvest streams (F_{b}, Fₕ), if present.

In embodiments, the growth process (captured through the parameter μ_{eff}) is modeled as the product of a growth rate under ideal conditions μₘₐₓ (where growth rate is assumed to be maximized), and one or more factors that describe the influence of other system variables on growth. These factors may in some cases take one of three functional forms: substrate limited ηₛ, quadratic η_{q} or inhibitory ηᵢ. The relationship between these factors and the resulting effective growth rate can be captured in equation (20):

μ_{eff}=μₘₐₓηₛη_{q}ηᵢ (20)

where the correcting factor ηₛ captures the contribution of Nₛ substrate limiting variables, η_{q} captures the contribution of N_{q} quadratic influencing variables and ηᵢ captures the contribution of Nᵢ inhibitory variables. Nₛ can be equal to 0 (no substrate limiting variable), 1 (one substrate limiting variable), or any natural number. N_{q} can be equal to 0 (no quadratic influencing variable), 1 (one quadratic influencing variable), or any natural number. Nᵢ can be equal to 0 (no inhibitory variable), 1 (one inhibitory variable), or any natural number. The correction factors above can be calculated as products of a plurality of correcting factors that each capture the contribution of a substrate limiting/quadratic influencing/inhibitory variable, as shown in equations (21)-(23) below:

ηₛ=Πₙ₌₁^{N}^{ₛ}(η_{s,n}) (21)

η_{q}=Πₙ₌₁^{N}^{_{q}}(η_{q,n}) (22)

ηᵢ=Πₙ₌₁^{N}^{ᵢ}(η_{i,n}) (23)

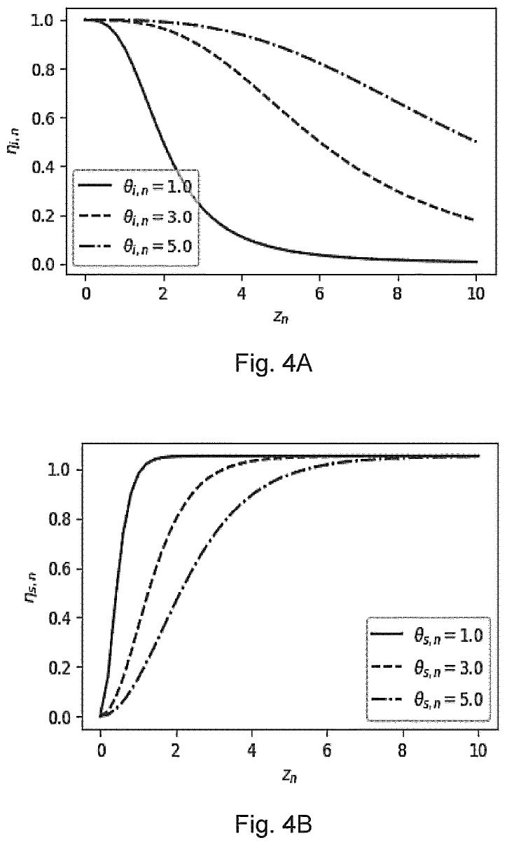

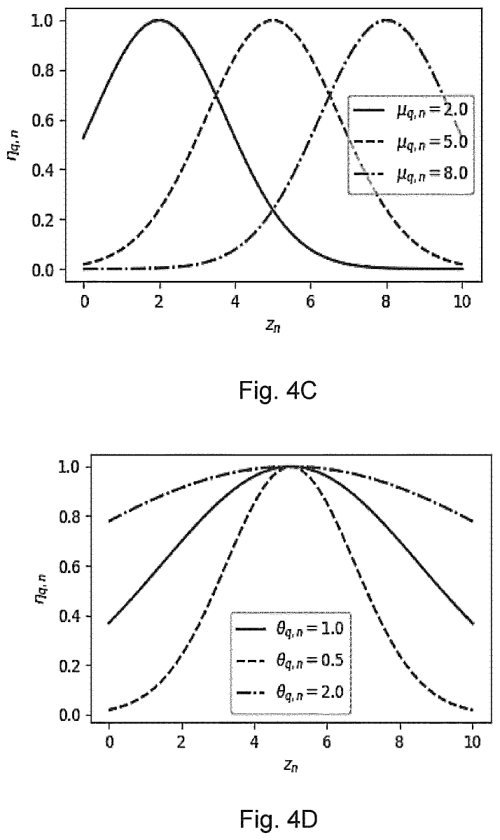

In embodiments, inhibitory effects are captured using correction factors of the form provided by equation (24) below:

$${η}_{i , n} = \frac{1}{{( \frac{{z}_{n}}{{θ}_{i , n}} )}^{3} + 1} \qquad ( 24 )$$

where zₙ is the concentration of a substance that has a growth inhibitory effect, and θ_{i,n} is a parameter that represents the level of zₙ above which inhibition occurs. FIG. 4A shows an example of the value of a growth inhibition factor as a function of the concentration of the growth inhibitory substance whose effect is modeled by the correction factor, for different values of the parameter θ_{i,n}. The variable ϕₜ in equations (18) and (19) above may be seen as a special case of an inhibitory effect where the concentration of the inhibitory substance depends on the viable cell density (e.g. because the inhibitory substance is produced by or as a result of the presence of the cells). The variable ϕₜ can also sometimes be modeled using a formulation as provided in equation (24). Therefore, equation 20) can in some embodiments be written as:

$${μ}_{eff} = {μ}_{max} {η}_{s} {η}_{q} {η}_{i} \frac{1}{{( \frac{{ϕ}_{t}}{{θ}_{t}} )}^{3} + 1} \qquad ( 25 )$$

where θₜ (or θ_{b} as the case may be) is a coefficient representing the inhibition in growth from accumulation of the biomaterial ϕₜ (where ϕₜ can be e.g. equal to xₗ or ϕ_{b}). As the skilled person understands, in some embodiments there may be more than one substance ϕ_{b}, each of which can be modeled using a respective term in equations (18) and (25) and a respective equation (19). Examples of substances that have a growth inhibitory effect and that produced by or due to the presence of the cells in the culture (i.e. substances ϕ_{b}) include toxic by-products such as e.g. ammonia. Examples of substances that have a growth inhibitory effect that is not necessarily related to the viable cell density include substances that are included in the culture medium to have a desired effect but that may also have a (ideally slight) growth inhibitory effect on the cell culture (e.g. antibiotics).

In embodiments, substrate limiting effects are captured using correction factors of the form provided by equation (26) below:

$${η}_{s , n} = tanh {( 2 \frac{{z}_{n}}{{θ}_{s , n}} )}^{2} \qquad ( 26 )$$

where θ_{s,n} is a parameter which represents the approximate level below which the variable zₙ (substrate that is growth limiting) starts to have a limiting effect on growth. FIG. 4B shows an example of the value of a substrate limitation factor as a function of the concentration of the limiting substrate whose effect is modeled by the correction factor, for different values of the parameter θ_{s,n}. In equation (26) the factor 2 is used to give the parameter θ_{s,n} an intuitive biological meaning as the approximate concentration zₙ below which a limiting effect can be seen (such as e.g. a value such that the inhibitory function crosses the ˜0.95 threshold when the value of zₙ falls below that of θ_{s,n}). Any other value can be used for this factor, resulting in the same behavior with the effect that the parameter θ_{s,n} is adjusted accordingly and no longer has the same intuitive interpretation. In particular, this factor can be omitted entirely (i.e. set to 1). Similarly, in equations (24) and (25), the term that is cubed (i.e. (zₙ/θ_{i,n})) may comprise a coefficient that gives the parameter θ_{i,n} an intuitive biological meaning as the approximate concentration zₙ above which a limiting effect can be seen (such as e.g. a value such that the inhibitory function crosses the ˜0.95 threshold when the value of zₙ reaches that of θ_{i,n}, for example 0.37). Examples of substances that have a substrate limiting effect include nutrients such as glucose, amino acids, etc.

In embodiments, quadratic effects are captured using correction factors of the form provided by equation (27) below:

$${η}_{q , n} = exp [ - \frac{1}{25} {( \frac{{z}_{n} - {μ}_{q , n}}{{θ}_{q , n}} )}^{2} ] \qquad ( 27 )$$

where μ_{q,n} is a parameter that represents the target value (i.e. the value at which maximum growth occurs) and θ_{q,n} is a parameter that represents the “spread” of the effect. The factor 1/25 used to give the parameter θ_{q,n} an intuitive meaning where a value of θ_{q,n}=1 means that the quadratic effect crosses a 95% threshold at ±1 around the target value μ_{q,n}. Any other value can be used for this factor, resulting in the same behavior with the effect that the parameter θ_{q,n} is adjusted accordingly and no longer has the same intuitive interpretation. In particular, this factor can be omitted entirely (i.e. set to 1).The use of factors that provide an intuitive interpretation of parameters such as θ_{s,n} and θ_{q,n} may be advantageous in that it may make it easier for a user to set these parameters (using e.g. biological knowledge or assumptions) or realistic boundaries for these parameters. FIG. 4C shows an example of the value of a quadratic effect factor as a function of the concentration of the substance whose effect is modeled by the correction factor, for different values of the parameter μ_{q,n} with a fixed value of θ_{q,n}=1. FIG. 4D shows an example of the value of a quadratic effect factor as a function of the concentration of the substance whose effect is modeled by the correction factor, for different values of the parameter θ_{q,n} with a fixed value of μ_{q,n}=5.

As the skilled person understands, other equations can be used instead or in addition to the above to model cell population variables and factors that affect these variables. These equations together may form a state space model (sometimes also referred to as a state observer). Indeed, they describe the evolution of the state of the cell culture (also referred to herein as “cell state”) in terms of a series of state variables xᵥ, x_{d}, xₜ, xₗ as a function of inputs to the system including e.g. the concentration of various substances that influence the cell state.

This state space model (comprising a kinetic growth model) can be augmented to include one or more equations that capture the evolution of the concentration of one or more metabolites in the bulk culture (including in particular nutrients, by-products and the desired product), as described above. Further, these equations can be used to calculate specific secretion/consumption rates. However, these equations only allow the calculation of the specific consumption/secretion rates at particular time points at which metabolite concentration measurements are available. As a result, the specific transport rates calculated using material balance equations can be used to identify that a fault has occurred but these equations do not enable the prediction of the value of the specific consumption/secretion rates at future time points. Thus, it is also not possible to calculate the concentration of the metabolites that can be expected at a future time point, since this requires the time dependent specific transport rates to be included in equation (4) (or equivalents). This in turn limits the ability to calculate the cell state variables that can be expected at such a future time point, since the concentration of metabolites that are substrate limiting, inhibitory or have a quadratic effect will influence the cell state variables (through the kinetic growth model as explained above). These problems are addressed by the present invention.

According to the invention, the specific transport rates of one or more metabolites of interest is predicted by a machine learning model. These predictions can be integrated into the state model described above by including them in equation (4) and equivalents, i.e. by augmenting the kinetic growth model described above with the following equations for one or more metabolites:

$$\frac{d ( {m}_{i} )}{dt} = \frac{{F}_{F}}{V} * {m}_{F , i} - \frac{{F}_{H}}{V} * {m}_{H , i} - \frac{{F}_{B}}{V} * {m}_{B , i} + {f}_{ML , i} ( u , m , s ) * {x}_{v} * ( \frac{{m}_{i}}{{m}_{i} + ε} ) \qquad ( 28 )$$

where ε can be set to 0 or a value that reflects the detection limit of the metabolite as explained above, and (u, m, s) in f_{ML,i} are values for the respective variables at one or more preceding time points. Such an augmented state model can be solved at every time point t to predict new values for mᵢ, xᵥ, x_{d}, and xₗ, which in turn can be used to predict new values for the specific transport rates at the next time point using the machine learning model, which prediction can be plugged back into the state model to predict new values for mᵢ, xᵥ, x_{d}, and xₗ, and so on and so forth.

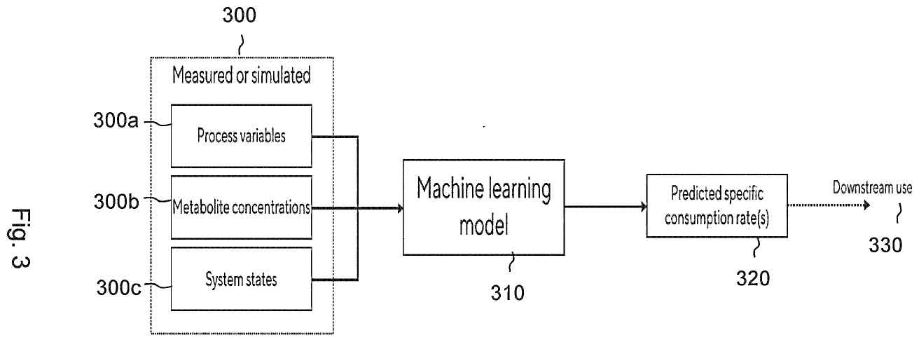

FIG. 2 is a flowchart illustrating a model calibration procedure, which can be used to obtain a calibrated machine learning model for predicting one or more metabolic condition variables of a bioprocess. At step 200, values for a plurality of process variables 200a and metabolite concentrations 200b (where the process variables and metabolite concentrations can collectively be referred to as process condition variables) associated with a bioprocess are received. These may have been measured using sensors (as will be explained further below), and/or inferred from measurements. Optionally, the values of one or more cell state variables 200c are also received. These may have been obtained by measurement and/or by inferring the values from measurements such as e.g. using a kinetic growth model as described herein. The values received at step 200 comprise input data for the machine learning model as well as data that can be used to verify the output of the machine learning model (i.e. labels or data from which labels can be calculated). At step 210, one or more of the values received are provided as input to a machine learning model which provides as output a set of predictions of specific transport rates in step 220. The predicted specific transport rates are compared to corresponding consumption rates obtained directly or indirectly from the measurements of step 200, at step 230. For example, the predicted rates may be compared to corresponding rates calculated using material balance equations as explained above. Alternatively, the predicted rates may be compared to corresponding measured values indirectly, by computing one or more further values based on the predicted rates and comparing the one or more values to corresponding measured values (or values derived therefrom). As an example, the predicted specific consumption/secretion rates may be used to calculate the concentration of one or more metabolites, and these concentrations may be compared to corresponding known values (i.e. concentrations that are measured or derived from measured values). Comparing the predicted and corresponding known values typically comprises calculating the value of a loss function as explained above. At step 240, the loss value calculated at step 230 is used by an optimization algorithm to modify the machine learning model. Steps 200, 210, 220, 230 and 240 are typically repeated using different or partially overlapping sets of input values received at step 200, all of which collectively constitute the training data. The process may be repeated until one or more stopping criteria are met. For example, stopping criteria may include a maximum number of iterations, a threshold on the amount of change in the value of the loss function compared to one or more preceding iterations, a target loss function value having been reached at one or more iterations, etc.

FIG. 3 is a flowchart illustrating a model deployment procedure through which metabolic condition variables of a bioprocess can be predicted. At step 300, values for a plurality of process variables 300a and metabolite concentrations 300b (where the process variables and metabolite concentrations can collectively be referred to as process condition variables) associated with a bioprocess are received. Optionally, the values of one or more cell state variables 200c are also received. The values received at step 300 are used at step 210 by a trained machine learning model to calculate one or more predictions of specific transport rates which are output at step 220. The predictions may then be used at step 330 for monitoring, controlling, optimizing or simulating a bioprocess. All of the values received at step 300 may have been measured using sensors, inferred from measurements, predicted by a model or set by a user (or a combination of these).

For example, in the context of process monitoring, some or all of the values may have been measured. Such measured values can be used by the machine learning model to predict specific transport rates that provide an indication of the future metabolic condition of the cells in the bioprocess that is being monitored. These predictions can also be used to calculate future metabolite concentrations and/or cell states, for example using material balance equations and/or a kinetic growth model as described herein. All of these features can each be used to determine whether the bioprocess is likely to operate normally for example using a multivariate model that has been parameterized to define a range of behaviours that are considered as “normal”, as described above. This can in turn be used to determine whether a corrective action should be taken, and also optionally what a suitable corrective action may be. Crucially, although multivariate approaches to identify bioprocesses that are operating “outside of specification” exist, their current implementation only allows to determine whether a bioprocess is currently operating normally. They do not allow the prediction of whether a bioprocess will operate normally at a future time point, or whether a bioprocess will operate normally if one or more process conditions are changed.

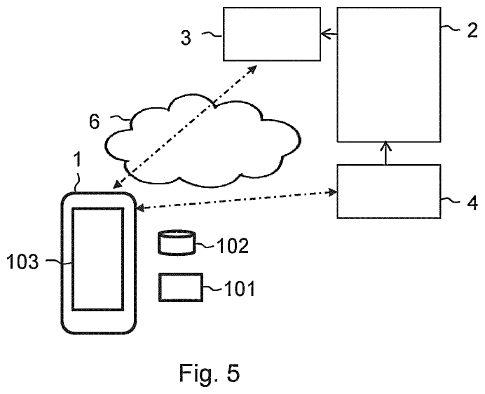

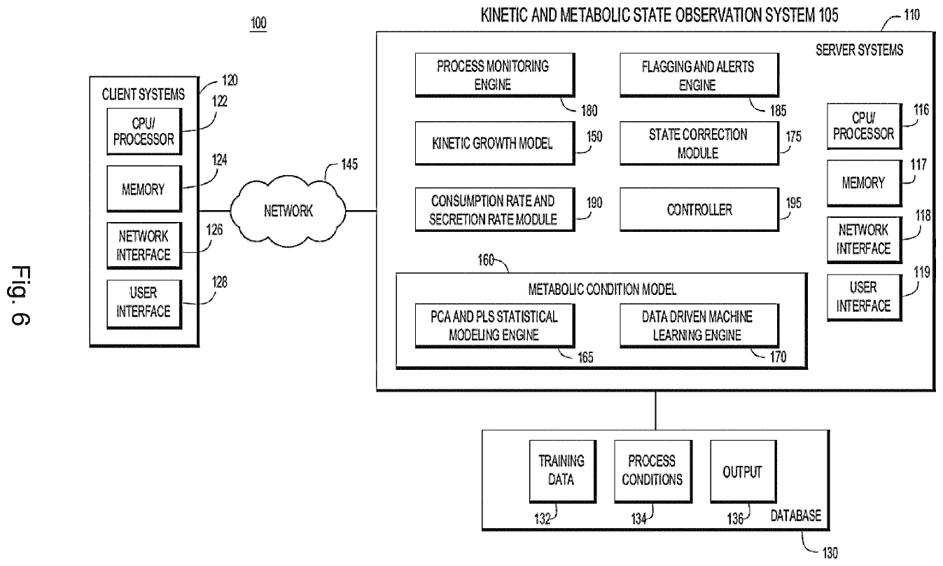

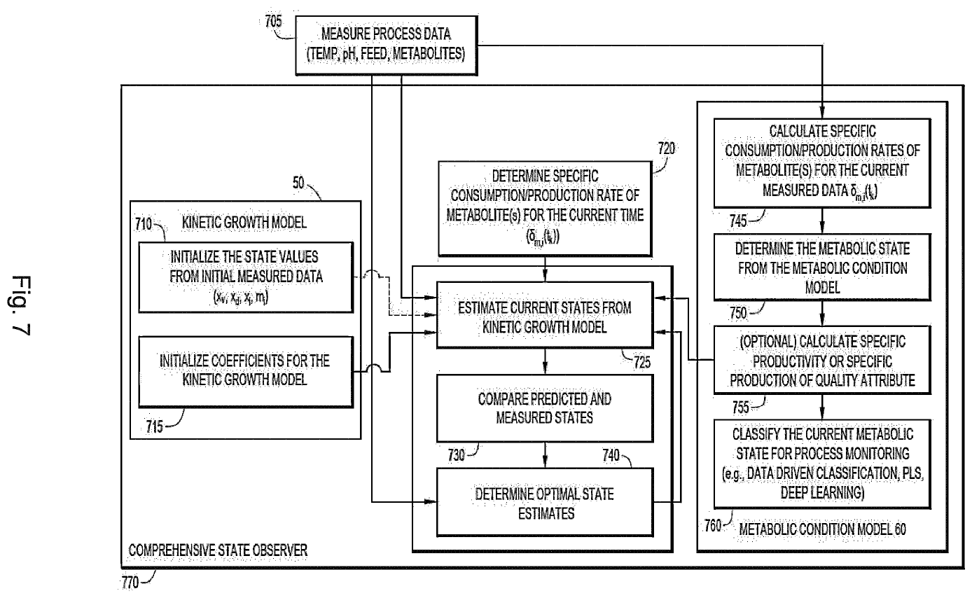

FIG. 5 shows an embodiment of a system for monitoring and/or controlling a bioprocess according to embodiments of the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is operably connected, such as e.g. through a network 6, to a bioprocess control system comprising a bioreactor 2, one or more sensors 3, and one or more effectors 4. The computing device may be a smartphone, tablet, personal computer or other computing device. The computing device is configured to implement a method for monitoring a bioprocess, as described herein. In alternative embodiments, the computing device 1 is configured to communicate with a remote computing device (not shown), which is itself configured to implement a method of monitoring a bioprocess, as described herein. In such cases, the remote computing device may also be configured to send the result of the method of monitoring a bioprocess to the computing device. Communication between the computing device 1 and the remote computing device may be through a wired or wireless connection, and may occur over a local or public network such as e.g. over the public internet. Each of the sensor(s) 3 and optional effector(s) 4 may be in wired connection with the computing device 1, or may be able to communicate through a wireless connection, such as e.g. through WiFi, as illustrated. The connection between the computing device 1 and the effector(s) 4 and sensor(s) may be direct or indirect (such as e.g. through a remote computer). The one or more sensors 3 are configured to acquire data that relates to a bioprocess being performed in the bioreactor 2, which can be implemented as illustrated on FIG. 1). The one or more effectors 4 are configured to control one or more process parameters of the bioprocess being performed in the bioreactor 2.

The one or more sensors 3 may each be on-line sensors (sometimes also referred to as “inline sensors”), which automatically measure a property of the bioprocess as it progresses (with or without requiring a sample of the culture to be extracted), or off-line sensors (for which a sample is obtained whether manually or automatically, and subsequently processed to obtain the measurement). Each measurement from a sensor (or quantity derived from such a measurement) represents a data point, which is associated with a time (or corresponding maturity) value. The one or more sensors 3 comprise a sensor that is configured to record the biomass in the bioreactor 2, referred to herein as a “biomass sensor”. This is typically in the form of a viable cell density or parameter from which viable cell density can be estimated. The biomass sensor may record a physical parameter from which the biomass in the bioreactor (typically in the form of the total cell density or the viable cell density) can be estimated. For example, biomass sensors based on optical density or capacitance are known in the art. The one or more sensors further comprise one or more sensors that measure the concentration of one or more metabolites, referred to herein as “metabolite sensors”. A metabolite sensor may measure the concentration of a single or a plurality of metabolites (such as e.g. from a few metabolites to hundreds or even thousands of metabolites), in the culture as a whole, the culture medium compartment, the biomass compartment (i.e. the cells as a whole), or specific cellular compartments. Examples of metabolite sensors are known in the art and include NMR spectrometers, mass spectrometers, enzyme-based sensors (sometimes referred to as “biosensors”, e.g. for the monitoring of glucose, lactate, etc.), etc. Most of the commonly used metabolite sensors measure the concentration of a metabolite in the culture medium. As used herein, sensors 3 (such as e.g. metabolite and biomass sensors) may also refer to systems that estimate the concentration of a metabolite or the amount of biomass from one or more measured variables (e.g. provided by other sensors). For example, a metabolite sensor may in practice be implemented as a processor (e.g. processor 101) receiving information from one or more sensors (e.g. measuring physical/chemical properties of the system) and using one or more mathematical models to estimate the concentration of a metabolite from this information. For example, a metabolite sensor may be implemented as a processor receiving spectra from a near infrared spectrometer and estimating the concentration of metabolites from these spectra. Such sensors may be referred to as “soft sensors” (by reference to their “measurements” being obtained using a software rather than by direct measurement). The one or more sensors 3 further include one or more sensors that measure further process conditions such as pH, volume of the culture, volumetric/mass flowrate of material in/out of the bioreactor, medium density, temperature, etc. Such sensors are known in the art. Whether one or more sensors 3 that measure further process conditions are necessary or advantageous may depend on at least the operating mode and the assumptions made by the material balance module as will be explained further below. For example, where the bioprocess is not operated as an unfed-batch, it may be advantageous to include one or more sensors measuring the amount and/or composition of the flows entering and/or leaving the bioreactor. Further, where the material balance module does not assume a constant volume in the bioreactor, it may be advantageous to include a sensor that measures the volume of liquid in the bioreactor (such as e.g. a level sensor). The measurements from the sensors 3 are communicated to the computing device 1, which may store the data permanently or temporarily in memory 102. The computing device memory 102 may store a trained machine learning algorithm as described herein. The processor 101 may execute instructions to predict one or more specific transport rates using the trained machine learning model and the data from the one or more sensors 3, as described herein (such as e.g. by reference to FIG. 3). Note that a system as described by reference to FIG. 5 may also be used to train a machine learning model as described herein (such as e.g. by reference to FIG. 2).

In the context of process simulation, some of the values may have been measured and others may have been predicted or set by a user. For example, a user may want to investigate the impact of implementing a particular change in one or more process conditions on one or more metrics of bioprocess performance. For this purpose, one or more values may be set based on measured time courses and others may be set to represent the intended change. Alternatively, all of the values may be set to represent a set of conditions that is intended to be used. The values comprise at least initial conditions that are sufficient for the machine learning model to predict a first set of specific transport rates. These predictions can be used to calculate future metabolite concentrations and/or cell states, for example using material balance equations and/or a kinetic growth model as described herein. These values can be fed back to the machine learning model, optionally in combination with further process parameters that are intended to be set by the user. A new set of predictions can then be obtained as above, and the process can be repeated as many times as desired. As above, the predictions themselves may provide an indication of the metabolic condition of the cells in the bioprocess that is being simulated. Further, the metabolite concentrations and/or cell states calculated as part of the process simulation process for example using material balance equations and/or a kinetic growth model as described herein may also provide information that is indicative of the performance of the cell culture. This application may be referred to as a “digital twin” because the simulation replicates real process conditions in silico in order to understand their effects on cell culture performance. Such simulation processes can be useful for example to investigate the impact of a process condition (e.g. temperature profile, pH, dissolved oxygen profile, agitation, medium composition, flow parameters, etc.) on a bioprocess. In such cases the initial conditions may include all starting process conditions that are used by the machine learning model including concentrations of metabolites, and the process conditions at each subsequent time point (e.g. used as inputs to the machine learning model and/or used as parameters of the material balance/kinetic growth model) other than metabolite concentrations (which will be calculated by the model) may be set to those that are investigated.

In the context of process optimization, the above described simulation process may be integrated as part of an optimization process through which a plurality of process conditions or combinations thereof are investigated and compared in view of one or more desirability criteria. For example, a desirability criterion may include the concentration of a desired product or total amount of desired product produced over a predetermined amount of time.

As the skilled person understands, the accuracy of the predictions from any machine learning model depends on a combination of the data that has been used for training the model and the particular use of the model. For example, the machine learning model may provide more accurate predictions when the input data has characteristics in terms of time differences between a plurality of time points that are similar to at least some of the training data that has been used to train the model. As another example, a machine learning model that has been trained using training data that reflects a wide variety of process conditions may provide more accurate predictions for previously unseen process conditions than a machine learning model that has been trained using training data that captures a much narrower set of conditions. Thus, without wishing to be bound by theory, it is believed that for the purpose of predictive process monitoring (where the process is expected to run within normal conditions), a machine learning model trained using data that is representative of a (typically relatively narrow) set of normal conditions (conditions that are known to result in a process within specification) may be sufficient. Conversely, for the purpose of simulation or optimization, a machine learning trained using data that is representative of a variety of conditions is likely to perform better. However, note that such simulation or optimization is simply not possible in the absence of prediction, such that even an imperfect machine learning model may provide an advantage.

Specific embodiments that make use of the prediction of specific consumption/secretion rates as part of a hybrid model for bioprocess monitoring, control, simulation and optimization will now be described.

Hybrid Model for Predictive Bioprocess Monitoring, Control, Simulation and Optimization

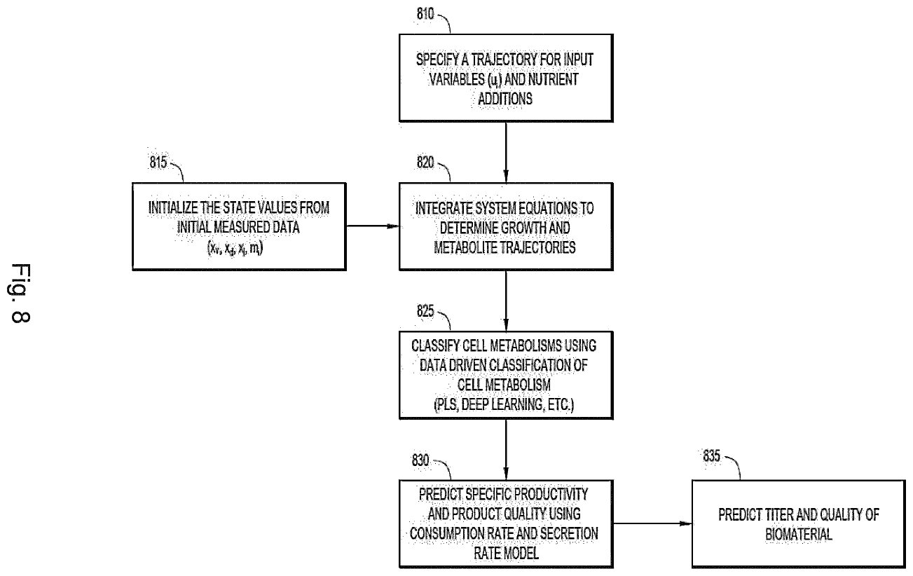

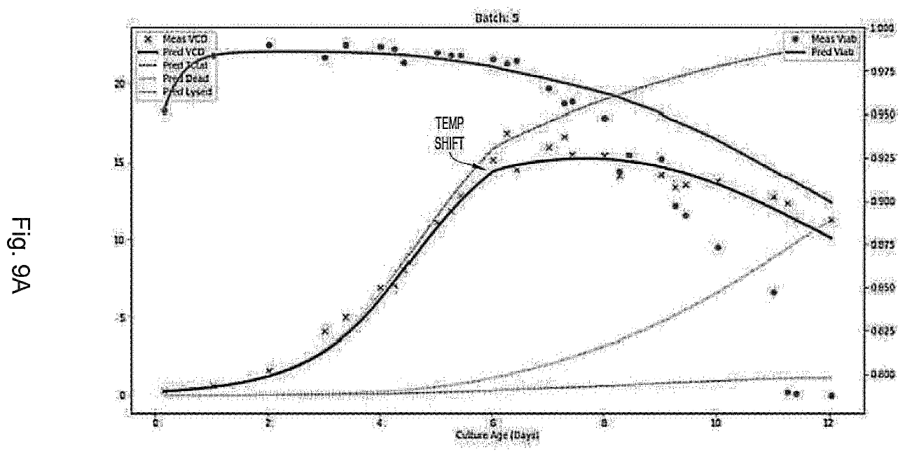

FIG. 6 shows an exemplary embodiment of a computing architecture for a hybrid model as described herein. A kinetic and metabolic state observation system 5 comprises a plurality of specific processing modules, including a kinetic growth model 50, a metabolic condition model 60, a state correction model 75, a process monitoring engine 80, a flagging and alerts engine 85, and a consumption rate and secretion rate module 90. The metabolic condition model 60 may comprise additional modules, including a multivariate statistical modeling engine 65 (also referred to herein as a PCA and PLS statistical modeling engine) and a data-driven machine learning engine 70. The kinetic growth model 50 and the metabolic condition model 60 together form a model referred to as a “hybrid model”. The kinetic model is a model designed to determine amounts of live cells, lysed cells, viable cells, and cell density, etc (as explained above). The metabolic condition model 60 is a model designed to provide product titer and quality control information about the titer and attributes (e.g., by-products, etc.) about the bioprocess. The output of the metabolic condition model 60 may feed into the kinetic growth model 50, i.e. some of the values calculated using the metabolic condition model 60 may be used as input variables in the kinetic growth model 50. The state correction model 75 may be used to update the estimate of the states of the kinetic growth model 50 based on experimental data. An error may be derived based on differences between measured experimental output and estimated hybrid model output, and the parameters of the hybrid model may be adjusted based on the error signal to drive the error signal to zero as a function of time.

Kinetic growth model 50 is a state space model based on the Monod growth equations (see equations (14)-(27)) and metabolite material balance equations (see equations (3)-(6) and (27)). The Monod growth equations and metabolite material balance equations are a series of differential equations that may be used to describe cell growth (e.g., microbial cell growth), cell density and viable cell density, total cells (e.g., viable cells, dead cells and lysed cells), etc. Inputs to the kinetic growth model may include temperature, feed conditions, pH, etc. and outputs include state estimates. The kinetic growth model may be seen as a classical state space model. In this context, parameters that are being modeled internally are referred to as states. For example, these parameters may include xᵥ, x_{d}, xₗ, mᵢ. The kinetic growth model may be used to monitor the number of live cells (and other parameters) in a bioreactor and to make predictions about the number of cells in the bioreactor at a future point in time. In addition to the kinetic growth equations (equations (14)-(27) above) and the material balance equations (e.g. equation (28) or any of equations (4) and equivalents), the kinetic growth model may comprise a separate equation to describe the rate of change of a desired biomaterial as a function of time. This can be represented as:

$$\frac{dIgG}{dt} = Qp ( m , {δ}_{m , i} ( t ) , u ) {x}_{v} \qquad ( 29 )$$

wherein

$$\frac{dIgG}{dt}$$

is the rate of change of the biomaterial as a function of time, Qp is a function with inputs comprising metabolite concentrations m, δ_{(m,i)}(t) is the specific production or specific consumption rate of a metabolite at the current time, u is a set of independent variables (i.e. variables that are provided as input to the model and for which the model does not calculate new values, for example these may include process parameters such as temperature, pH, etc.), and xᵥ is viable cell density. Where the biomaterial is itself a metabolite, equation (29) can take the form of a material balance equation as described above (and as such may be included as part of a set of material balance equations). Similarly, where the biomaterial is the biomass itself, equation (29) can take the form of the relevant kinetic growth equation, such as the equation capturing the viable cell density. In other words, equation (29) may already form part of the model as described above if the desired biomaterial is biomass or a metabolite already captured in the material balance equations included in the model. Outputs of the kinetic growth model include product titer, metabolite concentration, viable cell density, and viability. The kinetic growth model may also be used to calculate specific consumption (or secretion) rates for one or more metabolites the given time from measured data as explained above. According to the present invention, the specific consumption (or secretion) rate for one or more metabolites may be predicted for a future time using a trained machine learning model as described herein. Further, it is also possible for some specific transport rates to be calculated using the kinetic growth model and measured data, and for other specific transport rates to be predicted using a machine learning model.

Metabolic condition model 60 classifies the internal metabolic state of the system, e.g., into an optimal or suboptimal state or category for biomaterial production. Optionally, input into the metabolic condition model may include temperature, feed conditions, etc. In general, the metabolic condition model is independent from the kinetic growth model, and metabolic condition monitoring may be performed independently of kinetic growth. In embodiments, the metabolic condition model may be used to enhance the kinetic growth model by providing estimates of titer and/or quality to improve titer prediction. The metabolic condition model uses as minimal inputs the specific consumption/specific production of metabolites. As explained above, these may be calculated from metabolite concentrations and viable cell density data, or may be predicted using a machine learning model. The metabolic condition model optionally uses as input additional measured parameters and/or unmeasured states to improve prediction of product titer and/or quality. In some embodiments, the metabolic condition model comprises a statistical modeling engine 65 for building principal component analysis (PCA) or partial least squares (PLS) or orthogonal partial least squares (OPLS) models using the specific consumption/production rates (and optionally, additional parameters measured from the process or states) as inputs, and producing one or more multivariate scores as outputs. Statistical modeling engine may comprise any suitable engine, including (PCA) model, a partial least squares (PLS) model, a partial least squares discriminant analysis (PLS-DA) model, and/or an orthogonal partial least squares discriminant analysis (OPLS-DA) model. PCA may be used to characterize the variation within a data set, i.e. in this case to characterize metabolic variation. PLS may be used to correlate metabolic variation to productivity (titer) or production of an important quality metric (product quality). Techniques for performing PLS may be found, e.g., in Wold et al., PLS-regression: a basic tool of chemometrics, Chemometrics and Intelligent Laboratory Systems 58 (2001) 109-130. Techniques for performing PCA may be found, e.g., in Wold et al., Principal Component Analysis, Chemometrics and Intelligent Laboratory Systems 2 (1987) 37-52. Both PCA and PLS may be used to reduce dimensionality of a data set, i.e. to extract a set of summary variables that capture as much as possible of the variability in the data. PCA, a type of unsupervised dimension reduction technique, allows data to be summarized by linear combinations of variables without losing a significant amount of information. PLS, a type of supervised dimension reduction technique, is applied based on correlation between a dependent variable and independent variables. These techniques are considered to be within the scope of one of skill in the art.

In some embodiments, the output of the metabolic condition model 60 feeds into the kinetic growth model 50. The metabolic condition model 60 may also allow visualization of the metabolic condition of the cells in the bioprocess. Therefore, the output of the metabolic condition model 60 may serve as both an input to the kinetic growth model 50, as well as facilitates monitoring and visualization of the metabolic condition of the cell metabolism. The metabolic condition model may contain or be linked to a data-driven machine learning engine 70. Machine learning engine 70 may comprise one or more machine learning models such as a neural net, a deep learning model, or other machine learning model. Machine learning engine 70 may be trained using known techniques to classify the state of the biological system/bioreactor into an optimal or suboptimal state. Additionally, machine learning engine 70 may be trained using known techniques to classify the state of metabolite(s) into an optimal or suboptimal state. In embodiments, machine learning engine 70 performs classifications comparable to statistical modeling engine 65, and in some cases, with higher accuracy and precision than statistical modeling engine 65. The machine learning engine 70 further comprises one or more machine learning models trained to predict specific transport rates as described herein.

State correction model 75 mitigates error in the system. The difference between the output of the hybrid model and the measured data may be determined, and provided as an error signal to state correction model 75. The state correction model acts to minimize the error, and to drive the error signal to zero as a function of time. State correction may correspond to techniques associated with an extended Kalman filter. In embodiments, the state correction model 75 may be a separate module, or optionally, may be integrated into the kinetic growth model. Process monitoring engine 80 may interface with a plurality of sensors which measure one or more parameters associated with the bioreactor. These parameters may include temperature, oxygen levels, feed conditions, pH or other aspects of the bioprocess which may be monitored in real-time or in near real-time. These measurements may be provided to the hybrid model for simulating the bioreaction. These measurements may also be provided to the metabolic condition module 60 to monitor cell metabolism and generate state estimates. Flagging and alerts engine 85 monitors the system for process deviations. If an output of the bioreactor deviates from its expected/predicted output, an alert is provided to the user. In some aspects, the bioprocess may be suspended by the system until the process is corrected. In other aspects, the system may compensate for the deviation (e.g., adjusting the feed or process conditions to reach a desired state (e.g., an optimal state)). Flagging and alerts engine 85 may also send notifications to a user regarding the state of the bioreactor. In other aspects, a notification may be sent to a user, when the internal metabolic state is classified into a suboptimal category. Consumption rate and secretion rate module 190 determines the specific consumption rates and specific production rates of metabolites/analytes in the bioprocess reactor. The specific consumption data is used by the metabolic condition model 60. Outputs from the metabolic condition model 60 (for instance, estimation of specific productivity/specific consumption) may be provided into the kinetic growth model to predict product titer. Controller 95 may receive feedback (e.g., output of the bioreactor) to control the bioreactor to automatically adjust the experimental process conditions to minimize deviation from optimal process conditions.

Database 30 contains various types of data for the kinetic and metabolic state observation system 5. Training data 32 corresponds to data to identify kinetic model coefficients, calculate metabolite specific consumption/production rates, and/or train the metabolic condition model 60 to classify the state of the cells into an optimal or sub-optimal state or determine estimate the amount of a production of a biomaterial such as the specific productivity and/or to train the machine learning model that predicts specific consumption/secretion rates. Process conditions 34 correspond to the process conditions of the current bioprocess reaction. Process conditions 34 may also contain ideal process conditions that have been experimentally determined. These conditions may be supplied to the hybrid model (50, 60) to facilitate process monitoring of the current bioprocess operation or simulation/forecasting of the bioreactor. The output 36 is the output of kinetic and metabolic state observation system 5, and this output may be subtracted from the output of the experimental system in order to generate an error signal which is fed back into the input of the hybrid model.

Measurements of the metabolites and cell density may be obtained by process monitoring engine 80, and provided to the specific consumption and secretion rate model 190 to determine the specific consumption rate or the specific secretion rate of the metabolites, either by using the material balance equations as described above or by calling the machine learning engine 70. The specific consumption rate and specific secretion rate may be provided as input to the metabolic conditioning model. The specific consumption rate and specific secretion rate allow for translating process conditions into an amount each cell is consuming or producing for each of the metabolites (i.e. a metabolic condition variable). In embodiments, the specific consumption rate and specific secretion rate may be provided to the metabolic condition model 60, wherein the PCA and PLS statistical model engine 65 and/or data-driven machine learning engine 70 classifies the state of the cell to determine whether the system is in an optimal or a suboptimal condition, for example, with respect to a process parameter (e.g., temperature, feed concentration, pH, etc.).

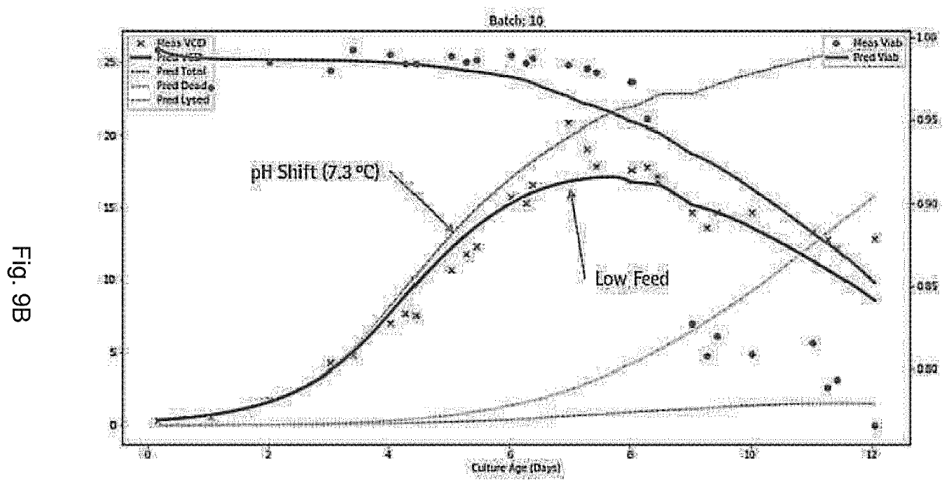

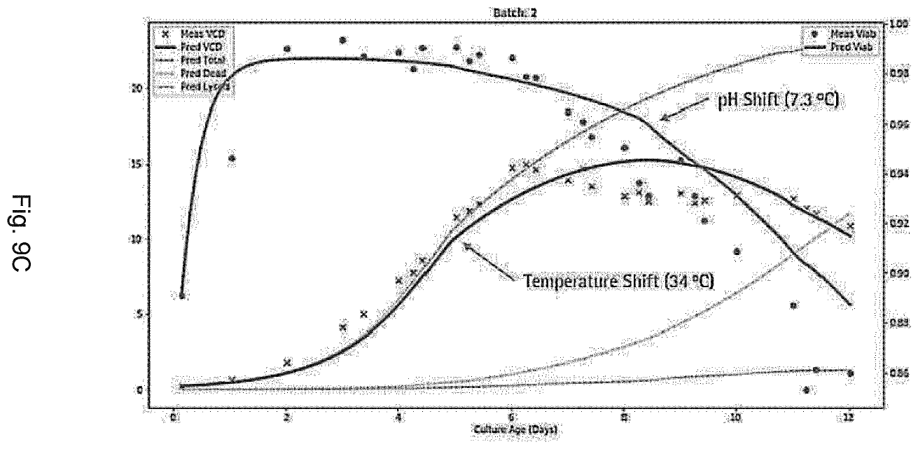

The architecture above may be used to monitor a bioprocess, as illustrated on FIG. 7. Using this architecture instead of simply using measurable characteristics of the bioprocess for monitoring purposes means that the internal states of the bioprocess (i.e. the characteristics of the cell culture and the metabolism of the cells) can be estimated, providing a richer picture of the state of the bioprocess. For example, a set of process parameters (also referred to as “inputs” or “output” of the bioreactor, depending on whether the parameters are set as operating conditions or whether they are results of the activity of the cells in the bioprocess) may be measured at step 705 and used as inputs to the hybrid model. These may include metabolite concentrations, viable cell densities (VCD), product titer, product quality, viability of cells, product quality, temperature, pH, dissolved oxygen (DO), etc. The process parameters are typically measured as a function of time. In embodiments, a zero-order hold is used to estimate the value over sampling intervals. The measured process parameters are provided to the kinetic growth model 50. The kinetic growth model can use some of this data (in particular, initial conditions) to initialize the state values (e.g., metabolite values), including xᵥ, x_{d}, xₗ, mᵢ at step 710. The kinetic growth model 50 can also be initialized with parameters at step 715 which may be provided by a user, retrieved as defaults or received together with the measured data. The kinetic growth model 50 can then be used to determine values for the states xᵥ, x_{d}, xₗ, mᵢ at step 725, simply by solving the equations in the model. This can be achieved by integrating the set of kinetic equations in the kinetic growth model from the time at the beginning of the reaction to the current time, given the measured values of process parameters. Solving the kinetic growth model requires knowledge of the specific consumption/production rates at every modeled time point. This knowledge can be obtained at step 720 from previous experiments, i.e. by calculating the rates from metabolite concentration and viable cell density measurements in previous experiments and assuming that these also apply to the present process (especially if the values obtained at corresponding times are used). Alternatively, one or more specific consumption/secretion rates can be predicted at step 720 using a machine learning model based on process parameters and/or kinetic growth model state estimates at previous time points, as described herein. The output of the kinetic growth model 50 may include product titer, metabolite concentration, viable cell density, and viability. As the skilled person understands, the kinetic growth model may include equations that capture the change in concentration of one metabolite, or a plurality of metabolites. Similarly, the specific consumption/secretion rates for one or a plurality of metabolites may be predicted and may be used by the metabolic condition model.

The kinetic growth model does not reflect the internal state of the cells in the bioreactor. If the cells experience fluctuations or deviations in process conditions (e.g., changes in temperature, increases or decreases in metabolites, feed condition alterations, etc.), the cells may enter a suboptimal state, and the output (e.g., titer of a biologic under production) may be suboptimal. Therefore, in embodiments, the kinetic growth model 50 may be supplemented with the metabolic condition model 60. This provides a way to estimate the internal state of the cell, and to correlate output production with environmental variables to optimize production. This may be particularly value in a multidimensional system having a large number of states, including some states that are correlated with each other, because in such cases determining which process condition(s) to adjust is challenging. Thus, metabolic condition model 60 provides a way to estimate the internal state of the cell, and to correlate output production with environmental conditions (variables) to optimize titer production. Further, if the system deviates from an optimal range, a user may receive a notification, prompting the user to correct the bioprocess to return the reaction to optimal conditions. In some embodiments, the system may autocorrect feed or environmental conditions to return the system to optimal conditions. For example, by providing feedback to a controller controlling the bioreactor, the experimental process conditions may be automatically adjusted to minimize deviation from the optimal process conditions. An optimization routine may further be used to determine process adjustments, as described below.

The functioning of the metabolic condition model 60 will now be described. The metabolic condition is determined at step 750 using the metabolic condition model, based on the specific consumption and production rates that have been calculated and/or predicted as explained above, at step 745. The metabolic condition may optionally further be used to calculate a specific productivity, or one or more quality attributes, at step 755. This may be done for example using (O)PLS models that have been trained to predict these values from the metabolic condition. The specific productivity (production of target protein) may be used in the kinetic growth model to estimate the product titer. For the purpose of process monitoring, the current metabolic condition can be classified at step 760 using one or more classification methods (such as e.g. machine learning classifiers or by comparing the current metabolic condition with a metabolic condition or range of conditions that are deemed to correspond to a normal and/or optimal state). Further, the data driven methods provided herein (e.g., PCA and PLS statistical modeling engine 65 and data driven machine learning engine 70) may be used to reduce the dimensionality of the system, and/or allow identification of conditions which impact titer. For example, when looking at a system having a high number of state variables (including e.g. a plurality of process conditions, metabolite concentration, growth variables and specific consumption/secretion rates) including variables that are correlated, it can be difficult to even identify deviations from optimality, let alone determine which process conditions to adjust in order to impact titer. The present techniques provide an improvement in the state of the art, as these techniques provide granular and specific control over the bioreactor, by identification of process variables that are outside an optimally determined range. In embodiments, optimal refers to a range for a process or feed condition that corresponds to optimized production of titer. However, any definition of optimality may be used.

The output of the hybrid model (e.g., state estimates, metabolic states, etc.) or of the kinetic growth model alone (in embodiments where a metabolic condition model is not included) may be compared with the output of the bioreactor at step 730, and the difference between the measured parameters and estimated parameters may be fed back through state correction model 75 into the input of the hybrid model 220, to improve the model. The state correction model 75 seeks to modify parameters to minimize the difference between the measured bioreactor output and the hybrid model output, to drive the error signal to zero as a function of time. In general, state estimators may be used to estimate the internal state of a system, when the state of the system is not directly measureable. In particular, Kalman filters may be used to determine an optimal estimate of the internal system states based on indirect measurements in a noisy environment, at step 740. That is, based on process conditions and kinetic models, a Kalman filter may be used to optimally estimate the internal state(s) of the system. Kalman filters are especially suitable for producing optimal estimates of system states in noisy systems. In this example, the state correction model 75 may comprise a Kalman filter or extended Kalman filter. The Kalman filter or extended Kalman Filter (EKF) may be used to determine optimal state values, wherein the error is combined with uncertainty in model state estimates, uncertainty in the state measurements, and covariance of the errors. Kalman filters may be applied to the kinetic growth model to improve the accuracy of the estimates of cell states provided by the model. The kinetic growth model and/or the metabolic condition model may be calibrated (a process which refers to the identification of suitable parameters in the models) using historical training data as known in the art.

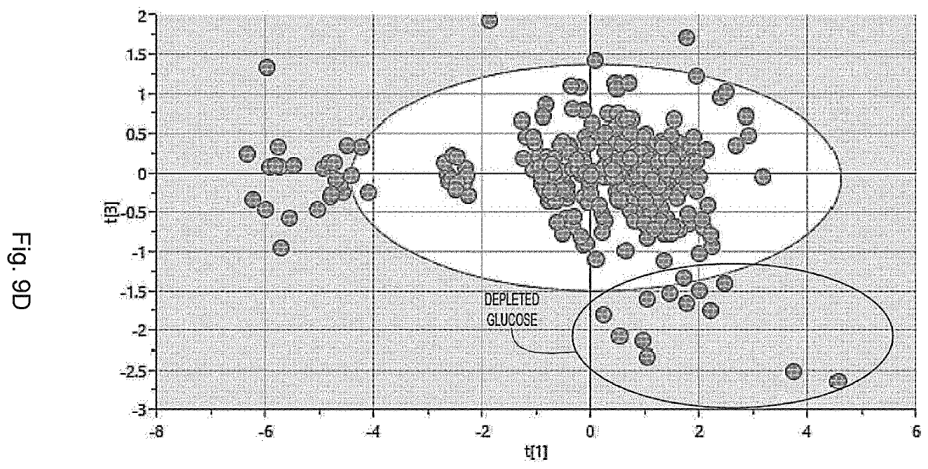

The hybrid model described above may also be used for optimization. Optimization may involve searching through various input variables to find a set that maximize titer, quality or other desired result, typically using an optimization algorithm. Optimization can be performed at run time, for example when monitoring a bioprocess as described above, in order to predict process conditions that may be used to restore an optimal state in a process that has been identified using the hybrid model to perform in a suboptimal manner. Optimization can also be performed independently of any particular run, such as e.g. to identify optimal process conditions for a future bioprocess. Input variables typically include nutrient additions and independent process parameters such as temperature and pH. An optimization process may proceed as described below, by reference to FIG. 8. A set of trajectories for input variables (uᵢ), which may include nutrient additions, is received at step 810. At step 815, the state values (i.e. the states of the kinetic growth model) are initialized from initial measured data (xᵥ, x_{d}, xₗ, mᵢ). The equations of the kinetic growth model are then integrated at step 820 to determine growth and metabolite trajectories over suitable time ranges. At step 825, the cell metabolic condition is determined using the metabolic condition model, and the metabolic condition is classified using data-driven classification (by the multivariate statistical modeling engine and/or the data driven machine learning engine). At operation 830, specific productivity and product quality are predicted using the metabolic condition model (e.g. using a multivariate model trained to predict titer from metabolic condition variables) and/or the kinetic growth model (e.g. by integrating the equation capturing the change in titer and/or quality of the product). At operation 835, titer and quality of biomaterial is predicted. This may be repeated using a different set of input variable trajectories and/or initial state values, in order to identify values of these variables that satisfy one or more optimality criteria. An optimization algorithm implements steps to explore a space of possible values in order to identify one or more sets of values that are satisfy these optimality criteria.

Finding the optimal adjustments or settings for a process may be defined as finding the set of manipulated variables (u or independent variables, in this case, process conditions and feeds) that minimize (or maximize) a mathematical objective function j. For example, an objective function to maximize the titer at a future time point t₍ₜ₊ₖ₎ may take the following form:

j=−I [symbol] |_{[t+k]′}=f_{IgG}(x|_{[t]}, u|_{[t])} (30)

where I [symbol] |_{[t+k]} is the predicted titer at the future time point, x|_{[t]} are the current value of the states, and u|_{[t]} are the set of manipulated variables to be implemented between now [t] and the future time point [t+k]. In embodiments, multiple objectives are simultaneously optimized. This may be performed by weighting the objectives based on their importance. For example, to maximize titer and maintain a quality metric on target, the objective function may take the following form:

j=−θ_{IgG}I [symbol] |_{[t+k]}+θ_{q}(\hat{q}|_{[t+k]}−qₛₚ)² (31)

where θ are the relative weights for each parameter to be optimized, and qₛₚ is the target or set point for the quality parameter q. There is a similar function (f_{q}) to the function for IgG to predict the future quality variable at a future time point. Further, constraints may be added to the function. For example, the optimization objective to maximize titer subject to maintaining quality within operating specifications may be governed by equation (31) subject to the following constraint:

qₘᵢₙ≤\hat{q}|_{[t+k]}≤qₘₐₓ (32).

Additionally, in order to prevent the optimization algorithm from selecting a new set of inputs that are infeasible, limits may also be placed on u. Additionally, the extent to which the optimization algorithm modifies conditions u when exploring the space of possible values or when providing an instruction to change current operating conditions to a controller (e.g. where a suboptimal state has been identified and the model has been used together with an optimization algorithm to identify changes to the process conditions that could correct this) can be tuned by placing penalties on changes in u from recipe or current settings. This prevents the controller from making erratic or large changes to the process conditions that provide minimal improvement to the objective parameters. The complete objective function then is described as finding the optimal set of feasible u that maximizes titer (IgG) and maintains quality on target and within specified limits. This may be governed by the following equation:

j=−θ_{IgG}I [symbol] |_{[t+k]}+θ_{q}(\hat{q}|_{[t+k]}−qₛₚ)²+θᵤ(u|_{[t]}−uₛₚ)²

subject to the constraint in equation (32) and uₘᵢₙ≤u|_{[t]}≤uₘₐₓ wherein θᵤ are the penalty weights for u (note there may be more than one u), and uₛₚ is the target value of u, which is usually a setpoint value or the current value.

The techniques provided herein provide models which accurately mimic cell behavior in a bioreactor. The predicted VCD and viability profiles were showed to match measured experimental values for various feed, pH, and temperature profiles, using experimental data. As can be seen on FIG. 9, the hybrid model described above was able to replicate experimentally measured behaviour, and to identify different functional cell states. In particular, as shown in FIGS. 9A-9C, a shift in temperature predicted by the kinetic growth model to inhibit growth was confirmed to do so. A pH shift appears to slightly increase cell death rates, but does not appear to inhibit growth rates. The cells seem to adapt and recover well from glucose and glutamine depletion. Therefore, no significant change is observed in growth, as the cells likely metabolize other carbon sources. As shown on FIG. 9D, cell state classification using a data-driven approach (PCA, in this case) applied to specific consumption rates calculated based on the measured data was able to identify that a bioprocess was operating in a suboptimal range.

Additionally, the hybrid model effectively acts as a soft sensor for cell metabolism and metabolites, allowing monitoring and characterization of metabolite specific consumption and production, as well as monitoring and characterization of changes in cell state and metabolic activity. Unlike other models, which do not estimate or otherwise account for lysed cells, the hybrid model takes into account the number of lysed cells, which influences bulk fluid toxicity. This approach allows the hybrid model to be more accurate than other models that do not account for this feature, and at longer time scales than other models. Other advantages of the hybrid model include having an increased knowledge of cell metabolism and of factors that drive cell growth, cell death, viability, titer and product quality. The hybrid model also provides the ability to simulate performance of new process conditions (e.g., feeding, temp, pH profiles, etc.) to maximize productivity and to observe cell state (e.g., metabolic activity, etc.) or changes thereof. In other aspects, perfusion performance may be predicted from fed batch operation. These techniques provide for improved forecasting, improved prediction of titer, and product quality based on monitoring and prediction. Present techniques are applicable to a wide variety of application areas including a simple univariate metabolite state estimator, a comprehensive multivariate metabolite state estimator, live systems (e.g., digital twin simulation), etc. Accordingly, present techniques are in an improvement in the field of bioreactor control and biologics manufacturing.

### EXAMPLES

Exemplary methods of calibrating a machine learning model, as well as exemplary methods for simulating a bioprocess will now be described.

Materials and Methods

Data

The data used in these examples was collected from 12 batches of cell cultures in miniature bioreactors. Each individual batch was allowed to run for 12 days. The cells in the bioreactor were CHO (Chinese Hamster Ovary) cells producing a recombinant antibody (denoted “IgG”). The concentration of this product is referred to as the “titer”. Over a period of 12 days that the batches are active the state of the bioreactor was measured 2-3 times a day (irregularly), yielding a little over 300 observations. A total of 11 independent variables were measured: viable cell density (VCD), cell viability (which may be obtained as VCD/TCD where the TCD is the total cell density, which may be equal to VCD+DCD where DCD is the dead cell density—in the present case the cell viability and VCD were obtained by measuring the TCD then counting cells in the presence of a dye, such as e.g. a fluorescent dye, that dyes live cells to obtain the VCD), dissolved oxygen (DO), pH, temperature, volume, and concentrations of glucose, glutamine, lactate, glutamate, and ammonia. Additionally, the titer was measured once a day, making the titer dataset only about a third the size of the metabolic dataset.

Missing values were linearly interpolated to keep as many observations as possible. Some titer measurements were poor and resulted in negative specific production rates, which is biologically implausible, so those titer measurements were removed.

The data for each measurement used for training was standardized (each observation X was scaled to

$$Z = \frac{X - μ}{σ}$$

where μ is the mean value of the variable and σ is the standard deviation). Standardization is expected to improve the speed and stability of the training as it reduces the risk of variables with large values having a disproportionate impact on the training because of their values, not because of their importance to the prediction. Standardization results in all variables Z being distributed with a mean=0 and a standard deviation=1. Standardization was performed only on the training data (see below), to reduce bias towards the validation data set.

Calculation of Labels

The term “label” refers to the (assumed) true value of a variable that is predicted by a machine learning model. In these examples, machine learning models were trained to predict specific consumption rates (SCRs) of a set of metabolites (δ_{m,i}) and the specific production rate (SPR) of a product (q_{IgG}), at a plurality of time points (tₖ). Thus, the labels for each of the plurality of time points are the (assumed) true value of these SCRs and SPRs. These were calculated for each time point using the equations below:

$${δ}_{m , i} ( {t}_{k} ) = ( {m}_{i , k} - {m}_{i , k + 1} + \frac{{mAdd}_{i , k}}{{V}_{k}} ) {iVCD}^{- 1} \\ {q}_{IgG} ( {t}_{k} ) = ( {C}_{IgG , k} - {C}_{IgG , k + 1} ) {iVCD}^{- 1} \\ iVCD = ( 0.6 {x}_{v , k} - 0.4 {x}_{v , k + 1} ) ( {t}_{k + 1} - {t}_{k} )$$

where m_{i,k} is the concentration of metabolite i at time k, mAdd_{i,k} is the bolus addition (fluid added to the reactor at discrete time-intervals to supply more nutrients) of metabolite i at time k, Vₖ is the total volume in the bioreactor, iVCD is the integrated Viable Cell Density, x_{v,k} is the viable cell density at time k, and C_{IgG,k} is the concentration of the product in the bioreactor at time k.

All labels were standardized across the training data set, as explained above. The final titer labels were calculated after filtering of the data to remove implausible values.

Machine Learning Models

In these examples, two machine learning models were independently trained: one for predicting the specific production rate of the product, and one for predicting the specific consumption/production rates of each of the measured metabolites (jointly). The former is referred to as “the titer network”, and the latter in referred to as “the metabolic network”. Both networks were feed-forward neural networks (NN). A variety of architectures were tested (data not shown) before choosing fully connected neural networks with three-hidden layers. A plurality of lag values (no lag, lag=1, lag=2) were tested, as well as a plurality of input variables.

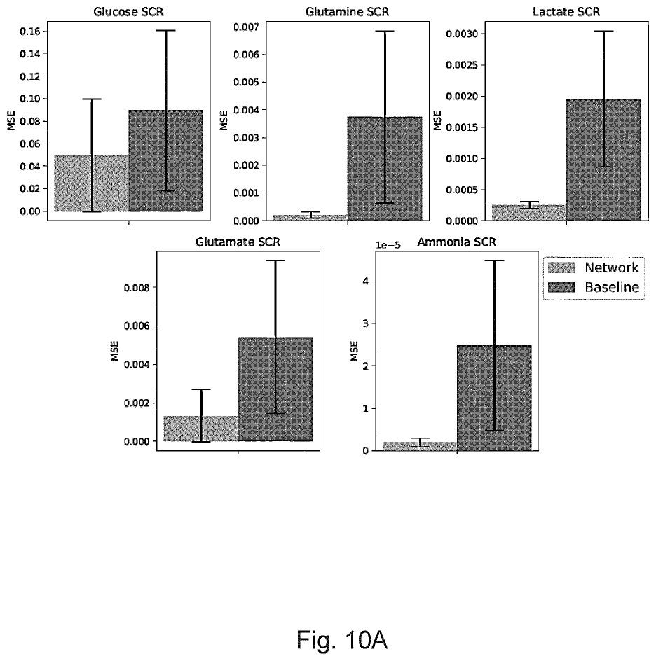

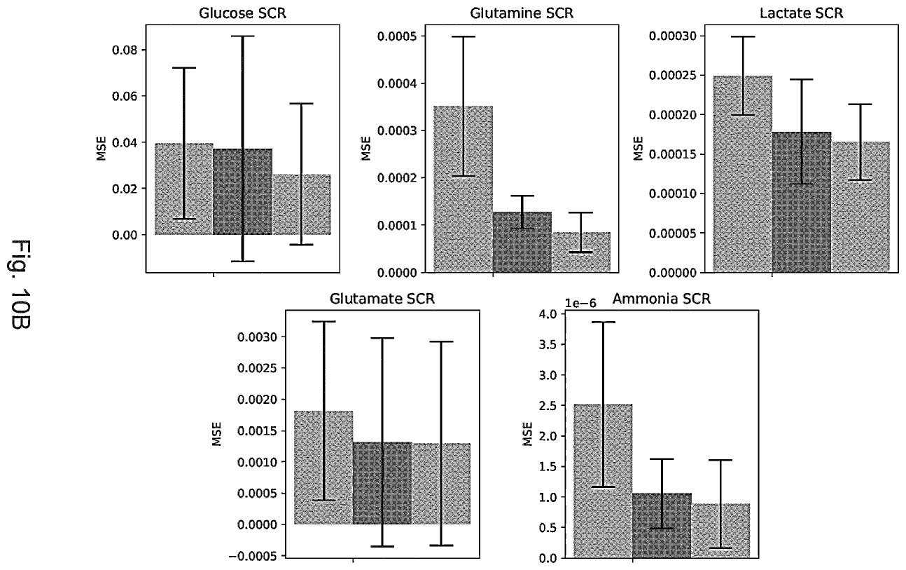

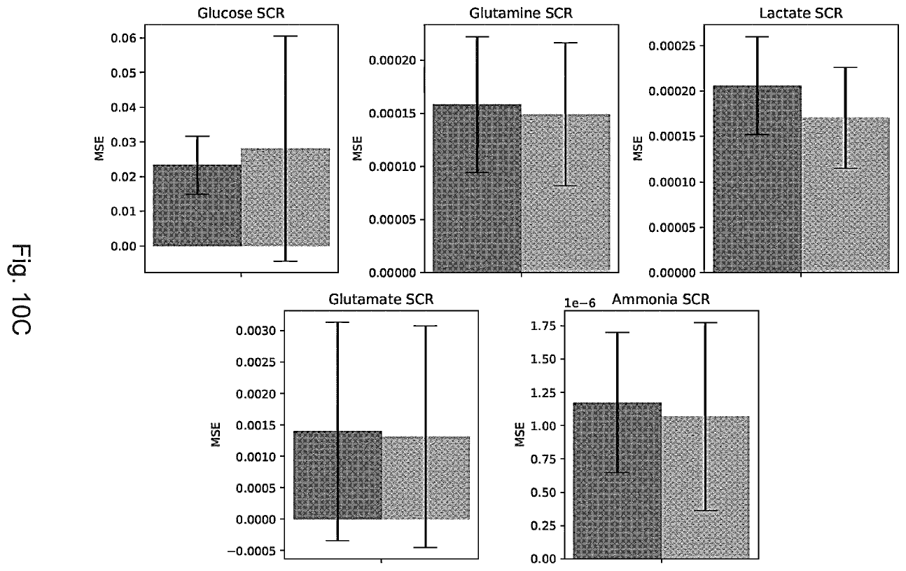

The final metabolic network uses lag=1 and predicts one-step-ahead (i.e. it predicts the value of one or more variables at time t—i.e. the specific consumption rate(s) between time point t and time point t+1—as a function of the values of one or more variables at time t and t−1). Indeed, the performance of the network with a lag=2 (two lagged values) was not found to be sufficiently better than with lag=1 in view of the increased computational time incurred. By contrast, a lag=1 was associated with a significant improvement in performance compared to a lag=0 (see FIG. 10B). The final metabolic network had 22 input nodes (11 variables, each provided for two time points), 22 nodes in each hidden layer, and 5 output values. Different sets of input variables were also tested to investigate the effects of using all variables available, vs. only the metabolite concentrations, or a subset of the metabolite concentrations. The results (see FIGS. 10C-D) indicated that the use of the metabolites concentrations alone (or even a subset of the metabolites concentrations) would likely be sufficient to obtain acceptable predictions in this case. However, as additional variables are expected to improve the predictions or at least their robustness, all available variables were used in the final network.

The final titer NN uses no lag values and predicts one-step-ahead (i.e. it predicts the value of one or more variables at time t—i.e. the specific consumption rate(s) between time point t and time point t+1—as a function of the values of one or more variables at time t). Indeed, a titer NN with lag=1 was compared to a titer value with lag=0 and these were not found to be significantly different (MES=123±116 for the titer NN without lag and 122±109 for the titer NN with lag of 1). AS the inclusion of a lag did not significantly improve performance, the simpler architecture was chosen for computational efficiency and ease of implementation in the bioreactor simulator.

ReLU was used as activation function for both networks.

Training of the Machine Learning Models

The training, or fitting, of the network is done by using a training dataset. This dataset contains input values and corresponding true output values, or labels. The input values are fed through the network and its output is compared to the labels by a loss function.

A loss function determines how close the network prediction was to the label, if the loss is 0, then the network prediction is the same as the label. A common loss function for regression problems is the mean squared error (MSE). This loss, or error, is then fed backwards through the network, calculating the gradients of the loss function with respect to the weights. This is called back-propagation of the error. Using the gradients the weights are then adjusted by an optimizer, typically based on gradient descent, to minimize the loss function.

Typically the network trains on the entire dataset many times, and each time it iterates through the dataset is called an epoch. Calculating the loss and gradients of the whole dataset can be very costly both in computation time and memory, so usually the loss is calculated on a small sample of the dataset, called a batch. Calculating the gradient and updating weights on a small sample of the dataset will result in a less accurate step toward minimizing the loss function for the entire dataset, but doing it many times will result in similar results while using less computing power resources and time.

By training a network for enough epochs, it is usually possible to reach a training loss of 0. However, this is usually a case of over-fitting. An over-fitted model does not generalize well to unseen data. In an effort to avoid over-fitting it is common to calculate the model's loss on a validation dataset, called the validation loss. The weights are not updated when going through the validation dataset. When the validation loss stops decreasing, it is usually a good idea to stop training the network to avoid over-fitting.

In the present examples, Adam (Diederik P. Kingma, Jimmy Ba, arXiv:1412.6980, December 2014) was used as optimizer, MSE was used as loss function, and the batch size was 5 for both networks. The metabolic NN was trained for 10 epochs with learning rate of 10⁻³ and then for 10 epochs with learn rate 10⁻⁴. The learning rate determines the speed of convergence of the optimization process by influencing the amounts that the weights in the neural network are updated during training. The learning rate is typically set by trial/error and/or based on experience. Decreasing the learning rate as the optimization progresses can allow to fine tune the weights of a networks when the optimization has converged to an area of the parameter space. The titer NN was trained for a maximum of 100 epochs or until no decrease in validation loss is observed, with a learning rate of 10⁻³ after which the model with the lowest validation loss was used. The training stopped after 30 consecutive epochs with no decrease in validation loss. Of note, such an “early stopping” approach would have also been suitable for training the metabolic NN.

Cross-Validation

The present examples use k-fold cross-validation, which is based on the idea of repeating the training and testing computation on different randomly chosen subsets of the original dataset. The procedure is such that a partition of the dataset is formed by splitting it into k non-overlapping subsets. The test loss is then estimated by calculating the average test loss across k trials. On trial i the i-th subset of the data is used as test set and the rest of the data is used for training.

In the present case, 6-fold cross-validation was used. The final labels dataset for the metabolic network includes 310 values, which were divided into a training set of size 258 and a validation set of size 52, at each iteration of the cross-validation procedure. The final labels dataset for the titer network includes 131 values, which were divided into a training set of size 109 and a validation set of size 22, at each iteration of the cross-validation procedure. To assign validation sets, observations from two complete batches were assigned as validation set instead of the standard practice assigning observations at random. Since observations within a batch are correlated, this strategy reduces risk of overly optimistic validation performance.

Evaluation—Monitoring

The predictions of the metabolic network were compared to the average SCR values at each time point across the 12 batches (29 values for each metabolite, over 12 days). This reflects the situation where, in the absence of the present method, the only way to predict the value of a specific consumption/production rate for a metabolite is by using a value derived from a set of batches that are assumed to follow a similar course.

In particular, the MSE between the predictions of the metabolic NN and the labels (see above) was calculated (for each validation data set in the cross-validation process), and the average MSE for each metabolite was used for comparison. This is referred to as the network MSE. Similarly, the MSE between the average SCR/SPR across 12 batches and the labels was calculated. This is referred to as the baseline MSE.

Evaluation—Simulation

Additionally, the metabolic network was used to predict specific consumption/production rates using simulated values obtained from a bioreactor model comprising a kinetic growth model as explained above, and material balance equations for each of the 5 metabolites for which data was available. The model was initialized with measured concentrations of the metabolites, then was allowed to run for 12 days using measured values of VCD, pH, temperature, DO, viability, but exclusively predicted values of the metabolite concentrations. The values were predicted using the kinetic growth model and material balance equations with the respective SCR/SPR in the material balance equations provided by the metabolic NN (and conversely the concentrations of the metabolites obtained by solving the kinetic growth and material balance equations were used by the metabolic NN to predict the SCR/SPR at the next step).

The predictions were evaluated by comparing the metabolite concentrations predicted by the simulator to a similar simulator that used the average SCR/SPR values calculated from the data from the 12 batches (data not shown). This simulator used, for every time point, the average SCR/SPR value that precedes and is closets to the integration time limit.

Note that the above comparisons do not fully reflect the performance of the NN approach as the NN approach is in theory capable of predicting SPR/SCR values for a variety of set ups (e.g. different process conditions), whereas using the average values from previously acquired data assumes that the process conditions used for acquiring said data are at least very similar (preferably identical) to the process conditions that are being simulated. In other words, the latter approach is limited to simulating conditions that have already been seen, and would likely perform significantly worse when attempting to simulate unseen conditions.

### Example 1—Monitoring

In this example, metabolic and titer networks were trained as explained in the methods section, and the predictions of the networks were compared to the corresponding baseline values calculated from previously acquired data as explained above.

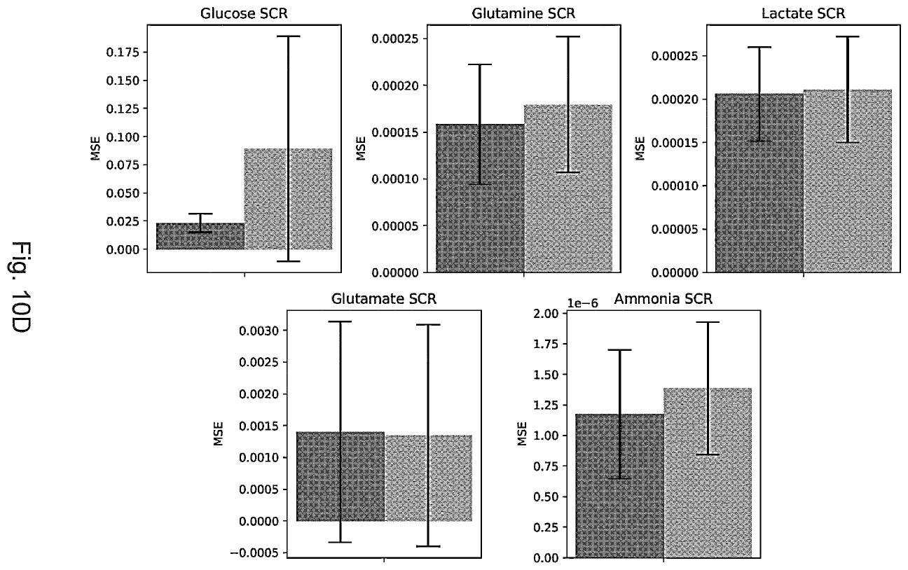

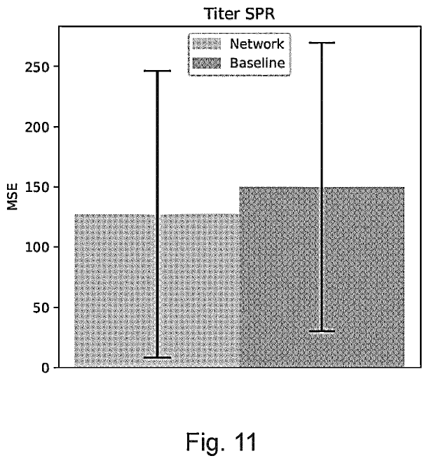

The results of this can be seen on FIGS. 10A and 11. On FIG. 10A, each plot compares: on the left, the MSE between the SCRs predicted by the metabolic NN for a metabolite and the corresponding labels (where the height of the bar represents the average MSE over the 6 folds of the cross-validation, and the error bars indicate the standard deviation across the 6 folds); and on the right, the MSE between the SCRs calculated as averages across 12 batches for a metabolite and the corresponding labels (where the height of the bar represents the average MSE over the 12 batches and the error bars indicate the standard deviation around this average).

Similarly, FIG. 11 compares: on the left, the MSE between the SPRs predicted by the titer NN and the corresponding labels (where the height of the bar represents the average MSE over the 6 folds of the cross-validation, and the error bars indicate the standard deviation across the 6 folds); and on the right, the MSE between the SPRs calculated as averages across 12 batches and the corresponding labels (where the height of the bar represents the average MSE over the 12 batches and the error bars indicate the standard deviation around this average).

As can be seen on FIG. 10A, the metabolic network MSE is significantly lower for every metabolite than the baseline MSE. FIG. 11 shows that the titer SPR predictions from the titer network have a slightly lower average MSE than the baseline. Note that the titer network used less data as input (due to the lag=0), and was only trained to predict a single value (the product SPR). By contrast, the metabolic network used a lag of 1 and was trained to jointly predict 5 values (the SCRs for each of 5 metabolites).

Metabolite SCR/SPRs are biologically related to each other through the metabolism of the cells. Therefore, it is plausible that jointly predicting values for multiple metabolites increases the performance of the network, if the network architecture is able to capture the correlations between those values that reflect their biology. Further, the product can be seen as a metabolite, such that the product SPR could have been predicted together with the metabolites SCR using a single network. This may increases the accuracy of the prediction, assuming that the network architecture is able to capture the correlations between the metabolites SCR/SPR that reflect the underlying biology.

FIG. 10B illustrates the benefits of increasing the lag values in the metabolic network, in terms of the MSE between the SCRs predicted by the metabolic NN for a metabolite and the corresponding labels (where the height of the bar represents the average MSE over the 6 folds of the cross-validation, and the error bars indicate the standard deviation across the 6 folds). In each plot, the bar on the left shows the MSE for predictions from a network trained with lag=0, the bar in the middle shows the MSE for predictions from a network trained with lag=1 (as in the final network used to produce the results on FIG. 10A), and the bar on the right shows the MSE for predictions from a network trained with lag=2. The data on FIG. 10B shows that increasing the lag from 0 to 1 is likely to be beneficial in this case, whereas the increase from lag=1 to lag=2 does not improve the prediction as much. FIG. 100 illustrates the performance of the metabolic network using all available variables (viable cell density (VCD), cell viability, dissolved oxygen (DO), pH, temperature, volume, and concentrations of glucose, glutamine, lactate, glutamate, and ammonia) as input (bar on the right, in each plot) vs. using only the 5 metabolite concentrations as inputs (bar on the left, in each plot), in terms of MSE calculated as described above. The data indicates that in this case the metabolite concentrations would have been sufficient to obtain useful predictions. FIG. 10D illustrates the performance of the metabolic network using only the 5 metabolite concentrations (bar on the left, in each plot—this corresponds to the bar on the left in each plot on FIG. 10C) vs. 4 out of the 5 metabolite concentrations (all apart from glucose; bar on the right, in each plot), in terms of MSE calculated as described above. The data indicates that the use of the metabolite concentrations for which a SCR is being predicted (or a closely related metabolite) is likely to improve the accuracy of the prediction for the particular metabolite. Indeed, the glucose SCR prediction appeared to be most affected by the absence of the glucose data, whereas other SCR predictions remained acceptable. As there did not seem to be a major downside to using all available variables, and the data indicates a trend towards lower MSE with additional variables, all were used in the final network (which was used to generate the data on FIG. 10A).

### Example 2—Simulation

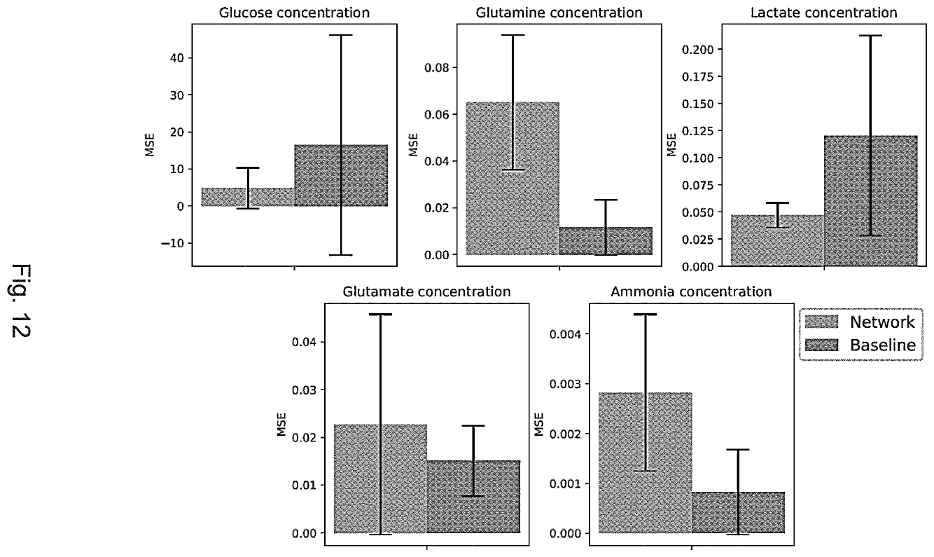

In this example, a metabolic network was trained as explained in the materials and methods section, and the predictions of the networks were used to simulate a bioreactor with initial conditions corresponding to the initial conditions in the data presented in the materials and methods sections, and process conditions other than metabolite concentrations and viable cell densities (which are predicted by the model) set to those in the data (i.e. pH, temperature, DO, volume). The predictions of the each simulation in terms of metabolite concentration were then compared to the corresponding measured concentrations by calculation of a MSE. The results of this are shown on FIG. 12.

On FIG. 12, each plot compares: on the left, the MSE between the concentration of a metabolite predicted by the model using the metabolic NN to provide the metabolite SCRs at each time point and the corresponding measured concentration (where the height of the bar represents the average MSE over the 12 batches, and the error bars indicate the standard deviation around this average); and on the right, the MSE between the concentrations of metabolites calculated by the same model but using the average SCRs calculated across 12 batches for a metabolite and the corresponding labels (where the height of the bar represents the average MSE over the 12 batches and the error bars indicate the standard deviation around this average).

As can be seen on FIG. 12, the metabolic network MSE is significantly lower for at least some of the metabolites than the baseline MSE. For the metabolites where the network simulation does not perform as well as the data-based simulation the difference in performance is relatively small considering that the data-based simulation compares measured metabolite concentrations with metabolite concentrations that have been obtained using a model that uses SCRs that were calculated using the very metabolite concentrations that the simulation results are compared to. Additionally, the network simulation MSE were always within less than an order of magnitude of the data-based MSEs (if not better). This is an acceptable level of error, considering that the network simulation opens up new avenues for simulation and optimization that would not be available for a data-based simulation, as explained above.

Equivalents and Scope

All documents mentioned in this specification are incorporated herein by reference in their entirety.

The terms “computer system” includes the hardware, software and data storage devices for embodying a system or carrying out a method according to the above described embodiments. For example, a computer system may comprise a central processing unit (CPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network.

The methods of the above embodiments may be provided as computer programs or as computer program products or computer readable media carrying a computer program which is arranged, when run on a computer, to perform the method(s) described above.

The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

Unless context dictates otherwise, the descriptions and definitions of the features set out above are not limited to any particular aspect or embodiment of the invention and apply equally to all aspects and embodiments which are described.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” or “approximately”, it will be understood that the particular value forms another embodiment. The terms “about” or “approximately” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

1. A method for monitoring a bioprocess comprising a cell culture in a bioreactor, the method comprising:
obtaining values of one or more process conditions including one or more process parameters, one or more metabolite concentrations and/or one or more biomass-related metrics for the bioprocess at one or more maturities;
determining the specific transport rates of one or more metabolites in the cell culture using the values obtained as input to a machine learning model trained to predict the specific transport rates of the one or more metabolites at a latest maturity of the one or more maturities or a later maturity based at least in part on the values of one or more process conditions for the bioprocess at the one or more maturities; and
predicting one or more features of the bioprocess based at least in part on the determined specific transport rates.

2. The method of claim 1, wherein predicting one or more features of the bioprocess comprises:
comparing the specific transport rates or values derived therefrom to one or more predetermined values; and
determining on the basis of the comparison whether the process is operating normally.

3. The method of any preceding claim, wherein the specific transport rate of a metabolite i is the net amount of the metabolite transported between the cells and the culture medium, per cell and per unit of maturity.

4. The method of any preceding claim, wherein the one or more process conditions include one or more process parameters selected from dissolved oxygen, dissolved CO2, pH, temperature, osmolality, agitation speed, agitation power, headspace gas composition (such as e.g. CO₂ pressure), flow rates (such as feed rate, bleed rate, harvest rate), feed medium composition and volume of the culture; wherein the one or more process conditions include one or more biomass related metrics selected from viable cell density, total cell density, cell viability, dead cell density, and lysed cell density; and/or wherein the one or more metabolite concentrations include the concentration of one or more metabolites in the cellular compartment, in the culture medium compartment, or in the cell culture as a whole.

5. The method of any preceding claim, wherein the one or more values of process conditions used to predict the specific transport rates of the one or more metabolites include at least one metabolite concentration value, preferably wherein the one or more values of process conditions further includes at least one further value of a process condition, preferably at least two further values, and/or wherein the one or more values of metabolite concentrations include the concentration of one or more metabolites for which specific transport rates are determined.

6. The method of any preceding claim, wherein predicting one or more features of the bioprocess comprising predicting the value of one or more critical quality attributes (CQAs) of the bioprocess using a predictive model that has been trained to predict CQAs using a set of predictor variables comprising the one or more specific transport rates.

7. The method of any preceding claim, wherein the machine learning model is a regression model, or wherein the machine learning model is selected from a linear regression model, a random forest regressor, an artificial neural network (ANN), and a combination thereof; and or wherein the machine learning model comprises a plurality of machine learning models, wherein each machine learning model has been trained to predict the specific transport rates of an individually selected subset of the one or more metabolites.

8. The method of any preceding claim, wherein the machine learning model has been trained to jointly predict the specific transport rates of the one or more metabolites at a later maturity based at least in part on the values of one or more process conditions for the bioprocess at one or more preceding maturities.

9. The method of any preceding claim, wherein obtaining values of one or more process conditions at one or more maturities comprises obtaining values of the one or more process conditions at a plurality of maturities; and the machine learning model has been trained to predict the specific transport rates of the one or more metabolites at a latest of the plurality of maturities or a later maturity based at least in part on the values of one or more process conditions for the bioprocess at the plurality of maturities, optionally wherein the machine learning model has been trained to predict the specific transport rates of the one or more metabolites at a latest of two distinct maturities or a later maturity based at least in part on the values of one or more process conditions for the bioprocess at the two distinct maturities.

10. The method of any preceding claim, wherein the values of one or more process conditions used as input to the machine learning model are associated with a plurality of maturities that are separated from each other by a difference in maturity that is approximately equal to the difference in maturity between the values used to train the machine learning model.

11. The method of any preceding claim, wherein predicting one or more features of the bioprocess comprises determining the value of one or more variables derived from the specific transport rates by: using the specific transport rates to determine the concentration of the corresponding one or more metabolites at the later maturity, optionally wherein determining the concentration of the corresponding one or more metabolites at the later maturity comprises solving respective material balance equations.

12. The method of claim 11, wherein determining the concentration of a metabolite i at maturity k, where k is the maturity associated with the predicted specific transport rates, comprises integrating any of equations (4), (4a)-(4f) and (28) between a preceding maturity at which mᵢ is known and maturity k.

13. The method of claim 11 or claim 12, further comprising determining the value of one or more variables derived from the specific transport rates by: using the specific transport rates to determine the concentration of the corresponding one or more metabolites at the later maturity, and using one of more of said concentrations to determine the value of a biomass related metric at the later maturity, optionally wherein determining the value of a biomass related metric at the later maturity comprises solving a kinetic growth model, optionally further comprising using one or more of the said metabolite concentrations and/or biomass related metric values as inputs to the machine learning model to predict specific transport rates at a further maturity.

14. The method of claim 13, further comprising predicting the effect of a particular value of a process parameter at a later maturity by including the particular value in the material balance equations, the kinetic growth model and/or the input values used by the machine learning model to predict specific transport rates at a further maturity.

15. A system for monitoring and/or controlling a bioprocess, the system including:
at least one processor; and
at least one non-transitory computer readable medium containing instructions that, when executed by the at least one processor, cause the at least one processor to perform the method of any of preceding claim;
optionally wherein the system further comprises, in operable connection with the processor, one or more of:
a user interface, wherein the instructions further cause the processor to provide, to the user interface for outputting to a user, one or more of: the value of the one or more specific transport rates or variables derived therefrom, the result of the comparison step, and a signal indicating that the bioprocess has been determined to operate normally or to not operate normally;
one or more biomass sensor(s);
one or more metabolite sensor(s);
one or more process condition sensors; and
one or more effector device(s).
